SEQUENCE LISTING (10 sequences)

SEQ ID NO: 1 - protein, 119 aa, Artificial Sequence
  Made in Lab - synthesized VH amino acid sequence
EVQLVESGGGLVKPGGSLKLSCAASGFKFSRYAMSWVRQAPGKRLEWVATISSGGSYIYYPDSVKGRFTISRDNVKNTLYLQMSSLRSEDTAMYYCARRDYDLDYFDSWGQGTLVTVSS

SEQ ID NO: 2 - protein, 107 aa, Artificial Sequence
  Made in Lab - synthesized VK amino acid sequence
DIQMTQSPSSLSASVGDRVTITCKASRDIRSYLTWYQQKPGKAPKTLIYYATSLADGVPSRFSGSGSGQDYSLTISSLESDDTATYYCLQHGESPFTLGSGTKLEIK

SEQ ID NO: 3 - DNA, 357 bp, Artificial Sequence
  Made in Lab - synthesized VH nucleotide sequence
gaagtgcagctggtggagtctgggggaggcttagtgaagcctggagggtccctgaaactctcctgtgcagcctctggattcaagtttagtagatatgccatgtcttgggttcgccaggctccggggaagaggctggagtgggtcgcaaccattagtagtggtggtagttacatctactatccagacagtgtgaagggtcgattcaccatctccagagacaatgtcaagaacaccctgtatctgcaaatgagcagtctgaggtctgaggacacggccatgtattactgtgcaagacgagattacgacctggactactttgactcctggggccaaggcacccttgtcaccgtctcctca

SEQ ID NO: 4 - DNA, 321 bp, Artificial Sequence
  Made in Lab - synthesized VK nucleotide sequence
gacatccagatgacccagtctccatcctccctgtctgcatcggtgggagacagagtcactatcacttgcaaggcgagtcgggacattagaagctatttaacctggtaccagcagaaaccagggaaagctcctaagaccctgatctattatgcaacaagcttggcagatggggtcccgtcgagattcagtggcagtggatctgggcaagattattctctcaccatcagcagcctggagtctgacgatacagcaacttactactgtctacaacatggtgagagtccattcacgctcggctcggggaccaagctggaaatcaaa

SEQ ID NO: 5 - protein, 10 aa, Artificial Sequence
  Made in Lab - synthesized heavy chain CDR1 amino acid sequence
GFKFSRYAMS

SEQ ID NO: 6 - protein, 17 aa, Artificial Sequence
  Made in Lab - synthesized heavy chain CDR2 amino acid sequence
TISSGGSYIYYPDSVKG

SEQ ID NO: 7 - protein, 10 aa, Artificial Sequence
  Made in Lab - synthesized heavy chain CDR3 amino acid sequence
RDYDLDYFDS

SEQ ID NO: 8 - protein, 11 aa, Artificial Sequence
  Made in Lab - synthesized light chain CDR1 amino acid sequence
KASRDIRSYLT

SEQ ID NO: 9 - protein, 7 aa, Artificial Sequence
  Made in Lab - synthesized light chain CDR2 amino acid sequence
YATSLAD

SEQ ID NO: 10 - protein, 7 aa, Artificial Sequence
  Made in Lab - synthesized light chain CDR3 amino acid sequence
LQHGESP

(19) United States
(12) Patent Application Publication
(10) Pub. No.: US 2022/0143140 A1
(43) Pub. Date: May 12, 2022

(54) ANTI-CD6 ANTIBODY COMPOSITIONS AND METHODS FOR TREATING LUPUS

(71) Applicants: Equillium, Inc., La Jolla, CA (US); University of Houston System, Houston, TX (US)

(72) Inventors: Stephen Connelly, La Jolla, CA (US); Krishna Polu, La Jolla, CA (US); Chandra Mohan, Houston, TX (US)

(21) Appl. No.: 17/434,247

(22) PCT Filed: Feb. 26, 2020
(86) PCT No.: PCT/US2020/019990

§ 371 Date: Aug. 26, 2021

Related U.S. Application Data
(60) Provisional application No. 62/810,628, filed on Feb. 26, 2019.
(60) Provisional application No. 62/933,294, filed on Nov. 8, 2019.

(51) Int. Cl. A61K 38/17; G01N 33/68; A61P 37/00; A61K 31/573; A61K 31/675; A61K 31/5377

(52) U.S. Cl. CPC A61K 38/1774; G01N 33/68; A61P 37/00; G01N 2800/52; A61K 31/675; A61K 31/5377; A61K 31/573

(57) ABSTRACT

The present disclosure provides methods of treating inflammatory or autoimmune diseases (e.g., lupus nephritis) using CD6-ALCAM pathway inhibitors such as EQ001 and to methods and diagnostic tests for identifying subjects likely to respond to such inhibitors. In particular, the present disclosure provides diagnostic and therapeutic uses related to elevated levels of soluble ALCAM and/or CD6 protein and protein fragments in urine and other biological samples that are indicative of sensitivity to inhibitors of the CD6-ALCAM pathway (e.g., EQ001).

### CROSS-REFERENCE

This application claims priority to U.S. Provisional Application No. 62/810,628, filed Feb. 26, 2019, and U.S. Provisional Application No. 62/933,294, filed Nov. 8, 2019, each of which application is incorporated by reference herein in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is EQIL_009_02WO_ST25.txt. The text file is 6 KB, was created on Feb. 26, 2020, and is being submitted electronically via EFS-Web.

### FIELD OF THE INVENTION

The present disclosure relates to, inter alia, methods for the treatment inflammatory or autoimmune diseases (e.g., lupus nephritis) using CD6-ALCAM pathway inhibitors such as an anti-CD6 antibody. Specifically, in some embodiments, this disclosure is concerned with methods of treating inflammatory or autoimmune diseases (e.g., lupus nephritis) in subjects that are identified as candidates for treatment with an anti-CD6 antibody (e.g., EQ001).

### BACKGROUND OF THE INVENTION

Inflammatory and autoimmune diseases are diseases are conditions that involve an abnormal response of the immune system to normal organ systems. The abnormal immune response can involve both innate and adaptive immune cell responses and can involve multiple cell types like T cells, B cells, dendritic cells, monocytes, and neutrophils. These diseases can be systemic affecting multiple organ systems or be confined to a single organ. The causes of these diseases are often unknown; they can run in families or be associated with environmental triggers or infections. Treatment for these diseases will vary by disease and involve therapies that broadly suppress the immune system or can target arms of the immune system including T cells, B cells, cytokines, and complement. While many different immune cells are involved in the pathogenesis of these inflammatory and autoimmune diseases, it is recognized that T cells play a central role in the initiation of the immune response and following inflammatory cascade. Further, T cells can also be pathogenic, traffic into tissues, secrete inflammatory cytokines, and recruit other inflammatory cells leading to tissue destruction and injury. There are over 80 autoimmune diseases with many of these diseases without FDA approved therapies. Given challenges with toxicities of drugs that suppress or modulate the immune system, biomarkers that can help identify patients most likely to responds to a given targeted therapy would have clinical utility in maximizing the benefit risk for population of patients with a given disease for a given therapy.

Lupus, a prototype of human systemic autoimmune disease, is characterized by a wide variety of multi-organ injuries. It is an autoimmune disease involving antibodies that attack connective tissue. The disease is estimated to affect nearly 1 million Americans, primarily women between the ages of 20-40. The principal form of lupus is a systemic one (systemic lupus erythematosus; SLE) and is associated with the production of anti-nuclear antibodies, circulating immune complexes, and activation of the complement system. While the pathogenesis of SLE is still not well understood, it is known that B cells, T-cells and monocytes are implicated in playing a critical role in the progression of the disease.

Specifically, there is a marked increase in polyclonal B-cell and T-cell activity and such increase can be characterized by the development of T-cells and antibody responses against a variety of self-antigens. It is theorized that the activation of T-cells stimulates the production of auto reactive B-cells to a specific epitope and then can spread to other epitopes. Such antibody response may include, as stated above, the production of autoantibodies against self-antigens such as anti-nuclear antibodies (ANA) and anti-double stranded DNA antibodies.

SLE can affect any organ system and can cause severe tissue damage. Untreated lupus can be fatal as it progresses from attack of skin and joints to internal organs, including lung, heart, and kidneys, with renal disease, termed lupus nephritis (LN), being the primary concern. Lupus mainly appears as a series of flare-ups (“active disease”), with intervening periods of little or no disease manifestation (“inactive disease”).

LN is one of the most acute areas of damage associated with pathogenicity in SLE, and accounts for at least 50% of the mortality and morbidity of the disease. LN is a heterogeneous disease involving multiple different immune cell types that drive immuno-pathogenesis. FIG. 1. Currently, there are no entirely curative treatments for patients who have been diagnosed with SLE or LN. From a practical standpoint, physicians generally employ a number of powerful immunosuppressive drugs such as high-dose corticosteroids, e.g., prednisone, or azathioprine or cyclophosphamide, which are given during periods of flare-ups, but may also be given persistently for those who have experienced frequent flare-ups. Even with effective treatment, which reduces symptoms and prolongs life, many of these drugs have seriously harmful side effects that require careful management. In addition, some patients are resistant or refractory to steroid treatments.

Further adding to the complexity of LN disease-management is the abovementioned dynamic nature of the disease. For example, LN immuno-pathogenesis dynamically progresses in patients through different stages of T then B cell-driven disease, and understanding when the disease pathogenesis is, e.g., more T-cell-driven may inform temporally when therapeutic intervention is most likely to succeed, as disease progression might be preventable early-on, when T cell are initiating an immune response. Further, understanding the underlying mechanisms of the pathology may inform on the types of treatments that are most likely to be effective, thus, enabling targeted therapy that treats the underlying disease, rather than merely nonspecifically turning off or down the entire immune system with general immune suppressants or chemotherapies.

Drugs targeting B cells alone have failed to show significant or consistent efficacy in the treatment of LN, and although T cell targeting therapies have shown benefit, their use are limited by toxicities that may be due to the heterogeneity of the disease and due to less than optimal treatment regimens due to lack of reliable information regarding whether diseases are active or inactive.

Liver biopsies may inform on LN disease status and progression to some degree, but they are limited to infrequent use, and because disease may not be evenly distributed over the sampled organ, they are susceptible to variable results. Blood markers may be suitable for some indications, but are not always indicative of the biology happening locally in the tissue.

As such, there remains a need for more effective treatments against LN with fewer harmful side effects. Additionally, there remains a need for ways of monitoring SLE and LN disease progression to effectively inform when a patient is in an active disease state or is transitioning into an active disease state from an inactive state. Further, there is a great need in the art for precision therapy methods for identifying patients that might be treatable with a particular therapy for SLE or LN, so that the need for utilizing the shotgun approach of treating with powerful immunosuppressive and chemotherapeutic agents may be reduced or eliminated. The present disclosure addresses these deficiencies.

### SUMMARY OF THE INVENTION

The present disclosure relates methods of treating inflammatory or autoimmune diseases or disorders (such as SLE or LN) in certain subsets of subjects that are determined to be candidates for a particular treatment. Specific embodiments relate to methods of treating inflammatory or autoimmune diseases or disorders (such as SLE or LN) in certain subsets of subjects that are determined to be candidates for treatment with an inhibitor of the CD6-ALCAM pathway.

In some embodiments, the present invention provides a method for identifying whether a subject has a form of lupus nephritis that is sensitive to CD6-ALCAM pathway inhibition, the method comprising determining whether the subject exhibits an elevated level of soluble CD6 and/or ALCAM protein.

In some embodiments, the present invention provides a method for treating lupus nephritis with a CD6-ALCAM pathway inhibitor, the method comprising:
(a) determining whether a biological sample obtained from a subject having or suspected of having lupus nephritis contains an elevated level of soluble CD6 and/or ALCAM protein; and
(b) administering to the subject a CD6-ALCAM pathway inhibitor if the biological sample contains an elevated level of soluble CD6 and/or ALCAM protein.

In some embodiments, the present invention provides a method for using a CD6-ALCAM pathway inhibitor to treat a subject with lupus nephritis, the method comprising the steps of:
(a) determining whether the subject exhibits elevated levels of soluble CD6 and/or ALCAM protein; and
(b) administering to the subject the CD6-ALCAM pathway inhibitor if the subject exhibits elevated levels of soluble CD6 and/or ALCAM protein.

In some embodiments, the present invention provides a method for treating a subject with a CD6-ALCAM pathway inhibitor, wherein the subject has lupus nephritis, the method comprising the steps of:
(a) determining whether the subject has a CD6-ALCAM pathway inhibitor-sensitive disease by:
(b) obtaining or having obtained a biological sample from the subject; and
(c) performing or having performed an assay on the biological sample to determine if the sample exhibits an elevated level of soluble CD6 and/or ALCAM protein; and
(d) administering the CD6-ALCAM pathway inhibitor to the subject if the subject has elevated soluble CD6 and/or ALCAM protein.

In some embodiments, the present invention provides a method for identifying whether a subject has an inflammatory or autoimmune disease that is sensitive to CD6-ALCAM pathway inhibition, the method comprising determining whether the subject exhibits an elevated level of soluble CD6 and/or ALCAM protein.

In some embodiments, the present invention provides a method for treating an inflammatory or autoimmune disease with a CD6-ALCAM pathway inhibitor, the method comprising:
(a) determining whether a biological sample obtained from a subject having or suspected of having inflammatory or autoimmune disease contains an elevated level of soluble CD6 and/or ALCAM protein; and
(b) administering to the subject a CD6-ALCAM pathway inhibitor if the biological sample contains an elevated level of soluble CD6 and/or ALCAM protein.

In some embodiments, the present invention provides a method for using a CD6-ALCAM pathway inhibitor to treat a subject with an inflammatory or autoimmune disease, the method comprising the steps of:
(a) determining whether the subject exhibits elevated soluble CD6 and/or ALCAM protein; and
(b) administering to the subject the CD6-ALCAM pathway inhibitor if the subject exhibits elevated soluble CD6 and/or ALCAM protein.

In some embodiments, the present invention provides a method for treating a subject with a CD6-ALCAM pathway inhibitor, wherein the subject has inflammatory or autoimmune disease, the method comprising the steps of:
(a) determining whether the subject has a CD6-ALCAM pathway inhibitor-sensitive disease by:
(b) obtaining or having obtained a biological sample from the subject; and
(c) performing or having performed an assay on the biological sample to determine if the sample exhibits an elevated level of soluble CD6 and/or ALCAM protein; and
(d) administering the CD6-ALCAM pathway inhibitor to the subject if the subject has elevated soluble CD6 and/or ALCAM protein.

In some embodiments, the present invention provides a method for identifying whether a subject has a form of lupus nephritis that is sensitive to CD6-ALCAM pathway inhibition, the method comprising determining whether the subject exhibits an elevated level of a CD6 and/or ALCAM polynucleotide. In certain embodiments, the methods comprise determining the level of messenger RNA (mRNA) expression of a polynucleotide encoding an ALCAM or CD6 polypeptide.

In some embodiments, the present invention provides a method for treating lupus nephritis with a CD6-ALCAM pathway inhibitor, the method comprising:
(a) determining whether a biological sample obtained from a subject having or suspected of having lupus nephritis contains an elevated level of the method comprising determining whether the subject exhibits an elevated level of a CD6 and/or ALCAM mRNA and
(b) administering to the subject a CD6-ALCAM pathway inhibitor if the biological sample contains an elevated level of the CD6 and/or ALCAM mRNA.

In various embodiments, the terms “a CD6 mRNA” and “an ALCAM mRNA” are used herein respectively to refer to an mRNA polypeptide encoding for a CD6 or ALCAM polypeptide.

In some embodiments, the present invention provides a method for using a CD6-ALCAM pathway inhibitor to treat a subject with lupus nephritis, the method comprising the steps of:
(a) determining whether the subject exhibits elevated levels of a CD6 and/or ALCAM mRNA; and
(b) administering to the subject the CD6-ALCAM pathway inhibitor if the subject exhibits elevated levels of the CD6 and/or ALCAM mRNA.

In some embodiments, the present invention provides a method for treating a subject with a CD6-ALCAM pathway inhibitor, wherein the subject has lupus nephritis, the method comprising the steps of:
(a) determining whether the subject has a CD6-ALCAM pathway inhibitor-sensitive disease by:
(b) obtaining or having obtained a biological sample from the subject; and
(c) performing or having performed an assay on the biological sample to determine if the sample exhibits an elevated level of a CD6 and/or ALCAM mRNA; and
(d) administering the CD6-ALCAM pathway inhibitor to the subject if the subject has elevated level of CD6 and/or ALCAM mRNA.

In some embodiments, the present invention provides a method for identifying whether a subject has an inflammatory or autoimmune disease that is sensitive to CD6-ALCAM pathway inhibition, the method comprising determining whether the subject exhibits an elevated level of a CD6 and/or ALCAM mRNA.

In some embodiments, the present invention provides a method for treating an inflammatory or autoimmune disease with a CD6-ALCAM pathway inhibitor, the method comprising:
(a) determining whether a biological sample obtained from a subject having or suspected of having inflammatory or autoimmune disease contains an elevated level of a CD6 and/or ALCAM mRNA; and
(b) administering to the subject a CD6-ALCAM pathway inhibitor if the biological sample contains an elevated level of a CD6 and/or ALCAM mRNA.

In some embodiments, the present invention provides a method for using a CD6-ALCAM pathway inhibitor to treat a subject with an inflammatory or autoimmune disease, the method comprising the steps of:
(a) determining whether the subject exhibits elevated CD6 and/or ALCAM mRNA; and
(b) administering to the subject the CD6-ALCAM pathway inhibitor if the subject exhibits elevated CD6 and/or ALCAM mRNA.

In some embodiments, the present invention provides a method for treating a subject with a CD6-ALCAM pathway inhibitor, wherein the subject has inflammatory or autoimmune disease, the method comprising the steps of:
(a) determining whether the subject has a CD6-ALCAM pathway inhibitor-sensitive disease by:
(b) obtaining or having obtained a biological sample from the subject; and
(c) performing or having performed an assay on the biological sample to determine if the sample exhibits an elevated level of a CD6 and/or ALCAM mRNA; and
(d) administering the CD6-ALCAM pathway inhibitor to the subject if the subject has elevated level of CD6 and/or ALCAM mRNA.

In some embodiments, any one of the methods disclosed herein may comprise a CD6-ALCAM pathway inhibitor that is EQ001.

In some embodiments, any one of the methods disclosed herein may comprise a CD6-ALCAM pathway inhibitor that is an anti-CD6 antibody, or the antigen binding fragment thereof. In some embodiments, the anti-CD6 antibody, or the antigen binding fragment thereof, is a humanized antibody. In some embodiments, the anti-CD6 antibody, or the antigen binding fragment thereof, binds to domain 1 or 3 on CD6. In some embodiments, the anti-CD6 antibody, or the antigen binding fragment thereof, binds to domain 3 on CD6. In some embodiments, the anti-CD6 antibody, or the antigen binding fragment thereof, is selected from the group consisting of: EQ001, ALZUMAb, UMCD6 mAb, Itolizumab, T1h, an anti-CD6 antibody described on Table 1, and an anti-CD6 antibody disclosed herein. In some embodiments, the anti-CD6 monoclonal antibody is an antibody produced by secreting hybridoma IOR-T1A deposited with the ECACC as deposit No. ECACC 96112640; an antibody having the same sequence as said antibody produced by said secreting hybridoma; or an antibody having the same CDR sequences of said antibody produced by said secreting hybridoma. In some embodiments, the antigen binding fragment is selected from an Fv, Fab, CDR1, CDR2, CDR3, combination of CDRs, variable region, heavy chain(s), and light chain(s). In some embodiments, the anti-CD6 antibody, or the antigen binding fragment thereof, comprises one or more CDR sequence selected from SEQ ID NOS: 5-10. In some embodiments, the anti-CD6 antibody, or the antigen binding fragment thereof, comprises heavy and light chain variable regions comprising amino acid sequences as set forth in SEQ ID NOs: 1 and 2. In some such embodiments, SEQ ID NOs: 1 and 2 are encoded by nucleotide sequences of SEQ ID NOs: 3 and 4 respectively. In some embodiments, the anti-CD6 antibody, or the antigen binding fragment thereof, comprises a VH sequence that is at least 80%, 85%, 90%, or 95% identical to the amino acid sequence as set forth in SEQ ID NO: 1. In some embodiments, the anti-CD6 antibody, or the antigen binding fragment thereof, comprises a VK sequence that is at least 80%, 85%, 90%, or 95% identical to the amino acid sequence as set forth in SEQ ID NO: 2. In some embodiments, the anti-CD6 antibody, or the antigen binding fragment thereof, comprises a VH sequence that is at least 80% identical to the amino acid sequence as set forth in SEQ ID NO: 1 and a VK sequence that is at least 80% identical to the amino acid sequence as set forth in SEQ ID NO: 2.

In some embodiments, a sample selected from blood, serum, urine, sputum, CSF, BALF, and stool is analyzed for levels of soluble CD6 and/or ALCAM protein according to the methods disclosed. In some embodiments, such a sample exhibits an elevated level of soluble CD6 and/or ALCAM protein (e.g., an elevated level as compared to a prior sample from the patient or as compared to a normal non-diseased patient or a reference level of the proteins that is normally seen in non-diseased patients). In some embodiments, a urine sample exhibits an elevated level of soluble CD6 and/or ALCAM protein (e.g., an elevated level as compared to a prior sample from the patient or as compared to a normal non-diseased patient or a reference level of the proteins that is normally seen in non-diseased patients).

In some embodiments, in any one of the methods disclosed herein, the subject may have lupus nephritis. In some embodiments, in any one of the methods disclosed herein, a subject that has lupus nephritis has elevated levels of soluble CD6 and/or ALCAM protein is as compared to an individual that does not have lupus nephritis.

In some embodiments, in any one of the methods disclosed herein, a subject that has an inflammatory or autoimmune disease has elevated levels of soluble CD6 and/or ALCAM protein is as compared to an individual that does not have the inflammatory or autoimmune disease.

In some embodiments, in any one of the methods disclosed herein, a level of soluble CD6 and/or ALCAM protein is determined in a first and one or more second sample from the subject. In certain embodiments, the level of soluble CD6 and/or ALCAM protein is elevated in a second sample as compared to the level of soluble CD6 and/or ALCAM protein that was present in the first sample. In certain embodiments, such an elevated level of soluble CD6 and/or ALCAM protein in the second sample indicates active disease in the subject. In certain embodiments, a decrease in the level of soluble CD6 and/or ALCAM protein in the second sample indicates transition from an active disease to a passive disease in the subject.

In some embodiments, a threshold increase in the level of soluble CD6 and/or ALCAM protein in the second sample as compared to the first sample indicates transition from a passive disease to an active disease in the subject. In some embodiments, the level of soluble CD6 and/or ALCAM protein is not elevated in a second sample as compared to the level of soluble CD6 and/or ALCAM protein that was present in the first sample. In some embodiments, the level of soluble CD6 and/or ALCAM protein in the second sample indicates that the subject does not have lupus nephritis or any inflammatory or autoimmune disease. In some embodiments, the level of CD6 and/or ALCAM is measured in a plurality of second samples obtained from the subject over a time course of days, weeks, months, or years.

In some embodiments, in any one of the methods disclosed herein, the level of CD6 and/or ALCAM protein is detected using a method selected from single-plex ELISA; multiplex ELISA, bead-based immunocapture with FACs-based detection; bead-based immunocapture with ELISA-based detection; bead-based immunocapture with chemiluminescent-based detection; meso-scale diagnostic (MSD); quantitative western blot; high performance liquid chromatography (HPLC); and a combination thereof.

In some embodiments, in any one of the methods disclosed herein, the CD6 and/or ALCAM protein that is detected is a full length protein. In some embodiments, the CD6 and/or ALCAM protein that is detected is a fragment of the full length protein. In some embodiments, the fragment of the full length CD6 protein that is detected comprises the entire extracellular domain of CD6, or a portion of the extracellular domain of CD6.

In some embodiments, in any one of the methods disclosed herein, the method may comprise administering to the subject EQ001. In particular embodiments, the methods disclosed herein may comprise administering to the subject EQ001 if the subject is determined to have active disease in accordance with a method disclosed herein, and/or if it is determined in accordance with a method disclosed herein that the subject has a form of lupus nephritis or of an inflammatory or autoimmune disease that is sensitive to CD6-ALCAM pathway inhibition. In some embodiments, the methods further comprises administering an additional therapeutic agent. In some embodiments, the additional therapeutic agent is a steroid or an immunosuppressant. In some embodiments, the steroid is a corticosteroid. In some embodiments, the corticosteroid is prednisone. In some embodiments, the agent is selected from mycophenolate and cyclophosphamide.

In some embodiments, the present invention provides a method of predicting the prognosis of a subject with lupus nephritis, the method comprising the steps of:
(a) obtaining or having obtained a plurality of biological samples from the subject over a time course of days, weeks, months or years; and
(b) performing or having performed an assay on each of the biological samples to determine if there is a change over time in the level of soluble CD6 and/or ALCAM proteins that are present in the sample; wherein
   (i) if the sample exhibits an increase in the level of soluble CD6 and/or ALCAM protein over time, then the prognosis is determined to be poor;
   (ii) if the sample exhibits no change in the level of soluble CD6 and/or ALCAM protein over time, then the prognosis is determined to be neutral; and
   (iii) if the sample exhibits a decrease in the level of soluble CD6 and/or ALCAM protein over time, then the prognosis is determined to be good.

In some embodiments, the present invention provides a method of predicting the prognosis of a subject with an inflammatory or autoimmune disease, the method comprising the steps of:
(a) obtaining or having obtained a plurality of biological samples from the subject over a time course of days, weeks, months or years; and
(b) performing or having performed an assay on each of the biological samples to determine if there is a change over time in the level of soluble CD6 and/or ALCAM proteins that are present in the sample; wherein
   (i) if the sample exhibits an increase in the level of soluble CD6 and/or ALCAM protein over time, then the prognosis is determined to be poor;
   (ii) if the sample exhibits no change in the level of soluble CD6 and/or ALCAM protein over time, then the prognosis is determined to be neutral; and
   (iii) if the sample exhibits a decrease in the level of soluble CD6 and/or ALCAM protein over time, then the prognosis is determined to be good.

In some embodiments, the present invention provides a method of determining whether a subject has active lupus nephritis comprising
(a) determining a first concentration of soluble CD6 and/or ALCAM protein present in a sample from the subject;
(b) determining a second concentration, or average concentration, of soluble CD6 and/or ALCAM protein present in a similar sample from a control person, or a population of control persons, respectively, that do not have active lupus nephritis; and
(c) determining that the subject has active nephritis if the first concentration is greater than the second concentration.

In some embodiments, the present invention provides a method of determining whether a subject has active inflammatory or autoimmune disease comprising
(a) determining a first concentration of soluble CD6 and/or ALCAM protein present in a sample from the subject;
(b) determining a second concentration, or average concentration, of soluble CD6 and/or ALCAM protein present in a similar sample from a control person, or a population of control persons, respectively, that do not have active lupus nephritis; and
(c) determining that the subject has active nephritis if the first concentration is greater than the second concentration.

In some embodiments, the present invention provides a method of determining whether a subject has transitioned from inactive lupus nephritis to active lupus nephritis comprising
(a) determining a first concentration of soluble CD6 and/or ALCAM protein present in a first sample from the subject; wherein the first sample is obtained from the subject when the subject has inactive lupus nephritis;
(b) determining a second concentration of soluble CD6 and/or ALCAM protein present in one or more second samples from the subject; wherein each second sample is obtained from the subject after the first sample was obtained; and
(c) determining that the subject has active lupus nephritis or is transitioning into active nephritis if the second concentration of soluble CD6 and/or ALCAM protein is greater than the first concentration.

In some embodiments, the present invention provides a method of determining whether a subject has transitioned from inactive inflammatory or autoimmune disease to active inflammatory or autoimmune disease comprising
(a) determining a first concentration of soluble CD6 and/or ALCAM protein present in a first sample from the subject; wherein the first sample is obtained from the subject when the subject has inactive lupus nephritis;
(b) determining a second concentration of soluble CD6 and/or ALCAM protein present in one or more second samples from the subject; wherein each second sample is obtained from the subject after the first sample was obtained; and
(c) determining that the subject has active lupus nephritis or is transitioning into active nephritis if the second concentration of soluble CD6 and/or ALCAM protein is greater than the first concentration.

In some embodiments, any one of the methods disclosed herein may further comprise administering to the subject EQ001 if the subject has active LN or is transitioning into active LN. In some embodiments, the method further comprises administering an additional therapeutic agent. In some embodiments, the additional therapeutic agent is a steroid or an immunosuppressant. In some embodiments, the steroid is a corticosteroid. In some embodiments, the corticosteroid is prednisone. In some embodiments, the agent is selected from mycophenolate and cyclophosphamide.

In some embodiments, in any one of the methods disclosed herein, the CD6-ALCAM pathway inhibitor is an anti-CD6 monoclonal antibody that is administered by parenteral delivery.

In some embodiments, in any one of the methods disclosed herein, the CD6-ALCAM pathway inhibitor is an anti-CD6 monoclonal antibody that is administered with a pharmaceutically acceptable carrier.

In some embodiments, in any one of the methods disclosed herein, the anti-CD6 antibody is a humanized antibody.

It is to be understood that one, some, or all of the properties of the various embodiments described herein may be combined to form other embodiments of the present invention. These and other aspects of the invention will become apparent to one of skill in the art.

### BRIEF DESCRIPTION OF THE SEQUENCES

SEQ ID NO: 1: Amino acid sequence of EQ001 VH sequence.

SEQ ID NO: 2: Amino acid sequence of EQ001 VK sequence.

SEQ ID NO: 3: Nucleotide (DNA) sequence of EQ001 VH sequence.

SEQ ID NO: 4: Nucleotide (DNA) sequence of EQ001 VK sequence.

SEQ ID NO: 5: Amino acid sequence of EQ001 VH CDR1

SEQ ID NO: 6: Amino acid sequence of EQ001 VH CDR2

SEQ ID NO: 7: Amino acid sequence of EQ001 VH CDR3

SEQ ID NO: 8: Amino acid sequence of EQ001 VK CDR1

SEQ ID NO: 9: Amino acid sequence of EQ001 VK CDR2

SEQ ID NO: 10: Amino acid sequence of EQ001 VK CDR3

### BRIEF DESCRIPTION OF THE DRAWINGS

FIG. 1. Heterogeneity in lupus nephritis. Analysis of LN kidney biopsies highlights differences in infiltrating immune cell populations by patient.

FIG. 2 Identification of LN patients that respond to CD6 targeted therapies. Based on the intrinsic heterogeneity in LN, certain subsets of patients are identified that respond to CD6 targeted therapies more effectively than other patients.

FIG. 3 Expression of CD6 & ALCAM within renal tissue. FIGS. 3A-3D show de novo analysis of publically available (Arazi 2019) single cell RNA Seq data obtained from frozen renal tissue samples or cells obtained from urine samples from LN and control subjects. FIG. 3A shows CD6 and ALCAM expression across renal cell types. FIG. 3B shows CD6 and ALCAM expression in epithelial cells and infiltrating leukocytes isolated from renal biopsies obtained from LN and control subjects as well as CD6 and ALCAM expression in urine leukocytes collected from LN patients. FIG. 3C shows renal CD6 expression in samples obtained from LN patients at different stages of the disease as compared to CD6 expression in control samples from healthy patients. FIG. 3D shows the number of CD6-positive T cells, ALCAM-positive tubules, and ALCAM-positive macrophages in samples from healthy or LN patients.

FIG. 4 Elevation of urinary ALCAM levels in LN patients. FIG. 4A shows ALCAM levels (pg/ml) in urine samples obtained from patients with active LN, active non-renal SLE, inactive SLE, and heathy controls. FIG. 4B shows performance of urinary ALCAM protein detection as a biomarker for LN.

FIG. 5 Urine ALCAM protein levels from SLE patients of multiple ethnicities and diverse disease activities. FIGS. 5A-5D: Cr-normalized urine ALCAM was significantly elevated in active LN patients of multiple ethnicities and discriminated patients with diverse disease activities. FIGS. 5E-5G: Urinary ALCAM correlated well with several clinical parameters including SLEDAI, renal domains of SLEDAI and PGA. HC, healthy controls, ANR, active non-renal lupus; AR, active renal lupus.

FIG. 6 Renal expression of ALCAM (CD166) and CD6 are increased in SLE-diseased mice. Kidneys harvested from 6 month old MRL/lpr mice, which have nephritis, and control C57BL/6 mice, which do not have nephritis, were stained for ALCAM (CD166, red, FIG. 6A and FIG. 6B) and CD6 (red, FIG. 6C), along with other markers as indicated including the myeloid marker CD11b or the T cell markers CD6 and CD3. Macrophages infiltrating the glomeruli of MRL/lpr mice were ALCAM+ are indicated with white arrows in FIG. 6A. CD6+ T cell infiltration is indicated with white arrows in FIG. 6C). Images are representative of 3 mice per group.

FIG. 7 Study design of the experiments using the MRL/lpr model.

FIG. 8 CD6 blockade reduces disease and the number of activated renal-infiltrating T cells in a model of SLE. FIG. 8A shows longitudinal proteinuria as measured by uristix. FIG. 8B shows proteinuria as measured by urine albumin:creatinine ratio. FIG. 8C shows kidney function as measured by blood urea nitrogen (BUN) levels in terminal serum. FIG. 8D shows Kaplan-meier curves depicting survival by treatment group. FIGS. 8E-8F show lymphadenopathy. FIG. 8E shows assessment of lymphadenopathy by the average of the volume measurement of the left and right inguinal lymph nodes at termination. FIG. 8F shows assessment of lymphadenopathy by scoring lymph node swelling. FIG. 8G shows the frequency of kidney infiltrating immune cells and T cells at termination are reduced by anti-mCD6 treatment. FIG. 8H shows number of total, effector/memory (CD44+), and activated (CD25+CD69+) CD4 T cells in the kidney at termination. FIG. 8I shows number of total and effector/memory (CD44+) CD8 T cells in the kidney at termination. For all FIGS in FIG. 8, **** p<0.0001; *** p<0.001; ** p<0.01; * p<0.05.

FIG. 9. CD6 blockade decreases pathology in the kidney. Histological scoring of a glomeruli (FIG. 9A) and renal tubules (FIG. 9B) was conducted by a blinded pathologist, showing significant improvement in glomerular pathology. *** p<0.001; ** p<0.01; * p<0.05.

FIG. 10. CD6 blockade improves skin manifestations of SLE. FIG. 10A shows histological examination of skin tissue. FIG. 10B shows macroscopic scoring of skin lesions at termination.

FIG. 11. CD6 blockade decreases infiltrating lymphocytes in skin. Skin tissue sections from MRL/lpr mice were stained for macrophages (green), C3 (red), and IgG (orange).

FIG. 11A shows staining of samples from MRL/lpr mice treated with isotype control. FIG. 11B shows staining of samples from MRL/lpr mice treated with anti-mCD6 antibody. FIG. 11C shows staining of samples from MPJ healthy control mice.

FIG. 12. Treatment of SLE/Lupis Nephritis (LN) with CD6 blockade in accelerated mouse model of nephrotoxic serum nephritis (NTN). FIG. 12A shows the experimental treatment schedule. FIG. 12B shows histological sections of renal tissue from vehicle and anti-mCD6 antibody treated animals. FIG. 12C shows blinded scoring of glomerular sections for endocapillary proliferation, crescent, and deposits, as assessed by an experienced nephropathologist on a scale from 0-4. FIG. 12D shows blinded scoring of tubular sections for tubular casts and interstitial inflammation, as assessed by an experienced nephropathologist on a scale from 0-4. FIG. 12E shows longitudinal proteinuria as measured by uristix. FIG. 12F shows urine albumin:creatinine ratio. FIG. 12G shows serum blood urea nitrogen levels (right panel) at termination (Day 11). Data in FIGS. 12E-12G are representative of two independent experiments.

FIG. 13. CD6 blockade decreases renal cytokine levels in nephritis. FIG. 13A shows mRNA expression levels of VCAM in kidney tissue. FIG. 13B shows mRNA expression of CCL5/Rantes in kidney tissue. FIG. 13C shows protein levels of inflammatory cytokines in the kidneys as assessed by multiplex, flow cytometry-based detection.

FIG. 14. CD6 blockade reduces immune infiltration into renal tissue. Flow cytometry was performed on kidneys to assess the effect of anti-mCD6 treatment on immune cell infiltration. FIG. 14A shows relative numbers of immune cell accumulation (CD45+) in anti-CD6 treated mice vs both isotype and vehicle control mice. FIGS. 14B-14D show inflammatory myeloid cells. FIG. 14B shows relative numbers of monocytes (CD11b+). FIG. 14C shows relative numbers of inflammatory macrophages. FIG. 14D shows relative numbers of neutrophils. FIGS. 14E-14F show relative numbers of T cell populations. FIG. 14E shows relative numbers of CD3+ T cells. FIG. 14F shows relative numbers of activated CD4 (CD25+CD69+) T cells.

FIG. 15. The CD6-ALCAM pathway is active in the NZB W F1 and B6.Sle1yaa models of SLE. FIG. 15A shows ALCAM (normalized to urine Creatinine level) is increased in NZB/W F1 mice post-disease development (12 months) vs pre-disease (≤6 months). FIG. 15B shows ALCAM (normalized to urine Creatinine level) is increased in B6.Sley1aa mice post-disease development (6 months) vs pre-disease (≤3 months). FIG. 15C shows treatment of the NZB/W F1 female mice starting at 26 weeks (˜6 months) with anti-mCD6 decreases proteinuria, an important measure of renal function.

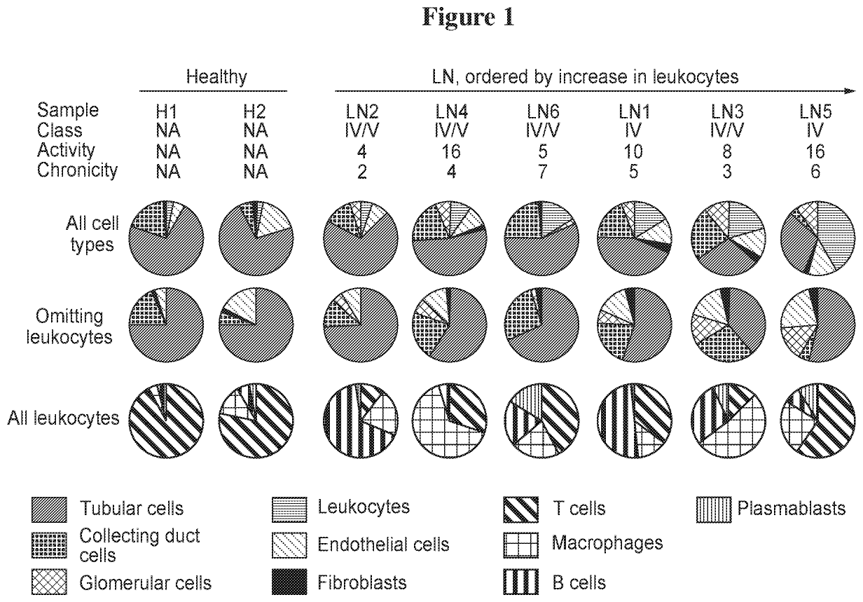

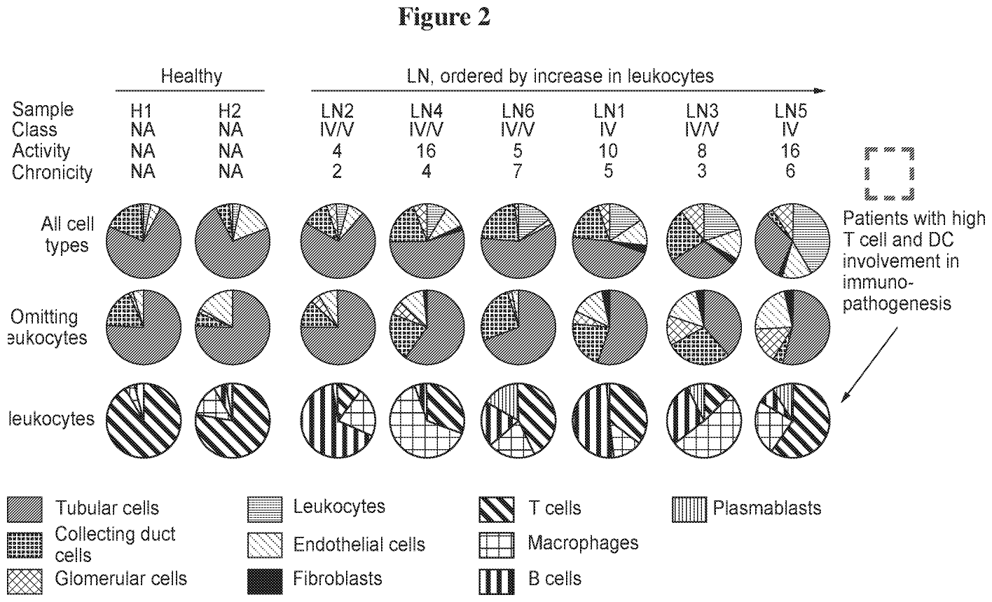

### DETAILED DESCRIPTION OF THE INVENTION

### General Methods

The practice of the present invention will employ, unless indicated specifically to the contrary, conventional methods of molecular biology and recombinant DNA techniques within the skill of the art, many of which are described below for the purpose of illustration. Such techniques are explained fully in the literature. See, e.g., Sambrook, et al., Molecular Cloning: A Laboratory Manual (3^{rd} Edition, 2000); DNA Cloning: A Practical Approach, vol. I & II (D. Glover, ed.); Oligonucleotide Synthesis (N. Gait, ed., 1984); Oligonucleotide Synthesis: Methods and Applications (P. Herdewijn, ed., 2004); Nucleic Acid Hybridization (B. Hames & S. Higgins, eds., 1985); Nucleic Acid Hybridization: Modern Applications (Buzdin and Lukyanov, eds., 2009); Transcription and Translation (B. Hames & S. Higgins, eds., 1984); Animal Cell Culture (R. Freshney, ed., 1986); Freshney, R. I. (2005) Culture of Animal Cells, a Manual of Basic Technique, 5^{th} Ed. Hoboken N.J., John Wiley & Sons; B. Perbal, A Practical Guide to Molecular Cloning (3^{rd} Edition 2010); Farrell, R., RNA Methodologies: A Laboratory Guide for Isolation and Characterization (3^{rd} Edition 2005). Poly(ethylene glycol), Chemistry and Biological Applications, ACS, Washington, 1997; Veronese, F., and J. M. Harris, Eds., Peptide and protein PEGylation, Advanced Drug Delivery Reviews, 54(4) 453-609 (2002); Zalipsky, S., et al., “Use of functionalized Poly(Ethylene Glycols)for modification of polypeptides” in Polyethylene Glycol Chemistry: Biotechnical and Biomedical Applications. The publications discussed above are provided solely for their disclosure before the filing date of the present application. Nothing herein is to be construed as an admission that the invention is not entitled to antedate such disclosure by virtue of prior invention.

### Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e., to at least one) of the grammatical object of the article. By way of example, “an element” means one element or more than one element.

The term “and/or” is used in this disclosure to mean either “and” or “or” unless indicated otherwise.

The term “e.g.” is used herein to mean “for example,” and will be understood to imply the inclusion of a stated step or element or group of steps or elements but not the exclusion of any other step or element or group of steps or elements.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

The term “administering”, as used herein, refers to any mode of transferring, delivering, introducing, or transporting matter such as a compound, e.g. a pharmaceutical compound, or other agent such as an antigen, to a subject. Modes of administration include oral administration, topical contact, intravenous, intraperitoneal, intramuscular, intranasal, or subcutaneous administration. Administration “in combination with” further matter such as one or more therapeutic agents includes simultaneous (concurrent) and consecutive administration in any order.

The term “binding partner” as used herein refers to matter, such as a molecule, in particular a polymeric molecule, that can bind a nucleic acid molecule such as a DNA or an RNA molecule, including an mRNA molecule, as well as a peptide, a protein, a saccharide, a polysaccharide or a lipid through an interaction that is sufficient to permit the agent to form a complex with the nucleic acid molecule, peptide, protein or saccharide, a polysaccharide or a lipid, generally via non-covalent bonding. In some embodiments the binding partner is a PNA molecule. In some embodiments the binding partner is an immunoglobulin or a proteinaceous binding molecule with immunoglobulin-like functions as defined below. In some embodiments the binding partner is an aptamer. In some embodiments a binding partner is specific for a particular target. In some embodiments a binding partner includes a plurality of binding sites, each binding site being specific for a particular target. As an illustrative example, a binding partner may be a proteinaceous agent with immunoglobulin-like functions with two binding sites. It may for instance be antigen binding fragment of an antibody. It may for instance be a bispecific diabody, such as a bispecific single chain diabody.

The term “carrier”, as used in this disclosure, encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

As used herein, the term “chimeric antibody” refers to an immunoglobulin polypeptide or domain antibody that includes sequences from more than one species. In a chimeric antibody a heavy chain or a light chain may contain a variable region sequence from one species such as human and a constant region sequence from another species such as mouse. As an example, a “chimeric antibody” may be an immunoglobulin that has variable regions derived from an animal antibody, such as a rat or mouse antibody, fused to another molecule, for example, the constant domains derived from a human antibody. The term “chimeric antibody” is intended to encompass antibodies in which: (i) the heavy chain is chimeric but the light chain comprises V and C regions from only one species; (ii) the light chain is chimeric but the heavy chain comprises V and C regions from only one species; and (iii) both the heavy chain and the light chain are chimeric.

An “effective amount,” when used in connection with a compound, is an amount of the compound, such as an anti-CD6 antibody (e.g., EQ001), needed to elicit a desired response. In some embodiments, the desired response is a biological response, e.g., in a subject. In some embodiments, the compound (e.g., an anti-CD6 antibody) may be administered to a subject in an effective amount to effect a biological response in the subject. In some embodiments, the effective amount is a “therapeutically effective amount.”

The terms “therapeutically effective amount” and “therapeutic dose” are used interchangeably herein to refer to an amount of a compound, such as an anti-CD6 antibody (e.g., EQ001), which is effective following administration to a subject for treating a disease or disorder in the subject as described herein.

The term “prophylactically effective amount” is used herein to refer to an amount of a compound, such as an anti-CD6 antibody (e.g., EQ001), which is effective following administration to a subject, for preventing or delaying the onset of a disease or disorder in the subject as described herein.

In this regard, a “humanized antibody” as used herein is an immunoglobulin polypeptide or domain antibody containing structural elements of a human antibody and the antigen binding site of a non-human antibody. “Humanized antibodies” contain a minimal number of residues from the non-human antibody from which they are derived. For instance, they may contain only the CDR regions of the non-human antibody, or only those residues that make up the hypervariable regions of the non-human antibody. They may also contain certain residues from outside the variable regions of the non-human polypeptide, such as residues that are necessary to mimic the structure of the non-human antibody or to minimize steric interference. Typically a humanized antibody contains a human framework, at least one CDR from a non-human antibody, with any constant region present being substantially identical to a human immunoglobulin constant region, i.e., at least about 85-90%, such as at least 95% identical. Hence, in some instances all parts of a humanized immunoglobulin, except possibly the CDRs, are substantially identical to corresponding parts of one or more native human immunoglobulin sequences. In addition, humanized antibodies may contain residues that do not correspond to either the human or the non-human antibodies.

As used herein, the term “antibody fragment” refers to any form of an antibody other than the full-length form. Antibody fragments herein include antibodies that are smaller components that exist within full-length antibodies, and antibodies that have been engineered. Antibody fragments include, but are not limited to, Fv, Fc, Fab, and (Fab′)2, single chain Fv (scFv), diabodies, triabodies, tetrabodies, bifunctional hybrid antibodies, CDR1, CDR2, CDR3, combinations of CDRs, variable regions, framework regions, constant regions, heavy chains, light chains, alternative scaffold non-antibody molecules, and bispecific antibodies. Unless specifically noted otherwise, statements and claims that use the term “antibody” or “antibodies” may specifically include “antibody fragment” and “antibody fragments.”

The term “VH” is used herein to denote the variable heavy chain of an antibody.

The term “VK” is used herein to denote the variable light chain of an antibody.

The term “Antigen binding fragment” in reference to an antibody refers to any antibody fragment that retains binding affinity for an antigen to which the parent full length antibody binds, and antigen binding fragments include, but are not limited to, Fv, Fab, (Fab′)2, scFv, diabodies, triabodies, tetrabodies, bifunctional hybrid antibodies, CDR1, CDR2, CDR3, combinations of CDRs, variable regions, heavy chains, light chains, and bispecific antibodies.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The term “modulating” includes “increasing,” “enhancing” or “stimulating,” as well as “decreasing” or “reducing,” typically in a statistically significant or a physiologically significant amount as compared to a control. An “increased,” “stimulated” or “enhanced” amount is typically a “statistically significant” amount, and may include an increase that is 1.1, 1.2, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the amount produced by no composition (e.g., in the absence of any of the anti-CD6 antibodies of the invention) or a control composition, sample or test subject. A “decreased” or “reduced” amount is typically a “statistically significant” amount, and may include a 1%, 2%, 3%, 4%, 5%, 6%, 7%, 8%, 9%, 10%, 11%, 12%, 13%, 14%, 15%, 16%, 17%, 8%, %19%, 20%, 25%, 30%, 35%, 40%, 45%, 50%, 55%, 60%, 65%, 70%, 75%, 80%, 85%, 90%, 95%, or 100% decrease in the amount produced by no composition (the absence of an agent or compound) or a control composition, including all integers in between.

The terms “polypeptide” and “protein” are used interchangeably herein to refer to a polymer of amino acid residues and to variants and synthetic analogues of the same. Thus, these terms apply to amino acid polymers in which one or more amino acid residues are synthetic non-naturally-occurring amino acids, such as a chemical analogue of a corresponding naturally-occurring amino acid, as well as to naturally-occurring amino acid polymers.

A “subject,” or “patient” as used herein, includes any animal that exhibits a symptom, or is at risk for exhibiting a symptom, which can be treated or diagnosed with an anti-CD6 antibody, or an antigen binding fragment thereof. Suitable subjects (patients) includes, preferably, human patients. Suitable subjects also include laboratory animals (such as mouse, rat, rabbit, or guinea pig), farm animals (such as pig, horse, cow), and domestic animals or pets (such as a cat or dog). Non-human primates (such as a monkey, chimpanzee, baboon or rhesus) are also included. In various embodiments, the terms “subject” and “patient” are used interchangeably.

“Substantially” or “essentially” means nearly totally or completely, for instance, 95% or greater of some given quantity.

“Treatment” or “treating,” as used herein, includes any desirable effect on the symptoms or pathology of a disease or condition, and may include even minimal changes or improvements in one or more measurable markers of the disease or condition being treated. “Treatment” or “treating” does not necessarily indicate complete eradication or cure of the disease or condition, or associated symptoms thereof. The subject receiving this treatment is any subject in need thereof. Exemplary markers of clinical improvement will be apparent to persons skilled in the art.

Unless defined otherwise, all technical and scientific terms used herein have the same meaning as commonly understood by one of ordinary skill in the art to which the invention belongs. Although any methods, compositions, reagents, cells, similar or equivalent to those described herein can be used in the practice or testing of the invention, the preferred methods and materials are described herein. All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

### Overview

The present disclosure relates to methods of treating inflammatory or autoimmune diseases or disorders (such as SLE or LN) in certain subsets of subjects that are determined to be candidates for a particular treatment (e.g., treatment with EQ001 and/or another inhibitor of the CD6-ALCAM pathway). In some embodiments, the present disclosure provides for the use of soluble CD6 protein, soluble ALCAM protein, or both as biomarkers indicative of active SLE, LN, or another inflammatory or autoimmune diseases or disorders disclosed herein or known in the art, and/or indicative of a patient's likelihood to respond favorably to a therapeutic intervention. The invention is based in part on the correlation of high soluble CD6 protein, high soluble ALCAM protein, or both with active T cell-driven inflammatory or autoimmune diseases or disorders (SLE or LN). High expression of one or both of these markers in a sample (e.g., a urine sample) from a patient that has, or is suspected of having, an inflammatory or autoimmune disease or disorder such as, e.g., SLE or LN may indicate increased signaling through the CD6-ALCAM pathway, which in turn informs that aberrant T-cell responses may underlie the active disease pathology.

CD6 is an important cell surface protein predominantly expressed by human T-cells and a subset of B-cells, as well as by some B-cell chronic lymphocytic leukemias and neurons. CD6 is a member of a large family of proteins characterized by having at least one domain homologous to the scavenger receptor cysteine-rich domain (SRCR) of type I macrophages. Blocking studies using anti-CD6 monoclonal antibodies (mAbs) suggest that CD6 plays an important role in T-cell development by regulating T-cell adhesive interactions with thymic epithelial (TE) cells.

Additional studies have shown that CD6 can function as an important accessory molecule in T-cell activation. For example, certain anti-CD6 mAb are directly mitogenic for T-cells [1, 2], whereas others are able to co-stimulate T-cell proliferation in conjunction with anti-CD3, anti-CD2 or phorbol 12 myristate 13 acetate (PMA) [1, 3, 4]. Yet additional evidence of the role of CD6 in T-cell activation comes from studies showing that CD6 becomes hyperphosphorylated on Ser and Thr residues [5, 6, 7] and phosphorylated on Tyr residues [8] following T-cell activation. These and other studies implicate CD6 as an important modulator of both immature and mature T-cell function in vivo, affecting both T-cell activation and signal transduction.

The extracellular domain of the mature CD6 protein is composed of three SRCR domains (hereinafter designated D1, D2, and D3). D3 corresponding to the membrane proximal SRCR domain followed by a short 33-amino acid stalk region. These extracellular domains are anchored to the cell membrane via a short transmembrane domain followed by a cytoplasmic domain of variable length [13].

A soluble form of CD6 (sCD6) of unknown origin has been reported to circulate at very low levels (pico/nano molar range) in sera from healthy individuals has been reported [14]. Further, elevated levels of sCD6 were observed in individuals with systemic inflammatory response syndrome [15] and primary Sjögren's syndrome [16], but direct mechanistic and functional relationships between these events are lacking. Reports suggest that sCD6 is formed by shedding of the membrane bound receptor via the proteolytic action of members of the ADAM family of metalloproteinases. Further, although the functional role of sCD6 in T-cell physiology is not yet known, in vitro results suggest that sCD6 inhibits T cell activation and maturation of the immunological synapse prompting some investigators to posit that sCD6 acts as a decoy receptor to inactivate bystander T cells near a site of inflammation.

Studies using CD6-immunoglobulin fusion proteins, containing selected extracellular domains of CD6 fused to human IgG1 constant domains (CD6-Rgs), led to the identification and cloning of a CD6 ligand, designated “activated leukocyte cell adhesion molecule” (ALCAM) also known as CD166 [9, 10].

ALCAM, is a 100-105 kD type I transmembrane glycoprotein that is a member of the immunoglobulin superfamily and comprises five extracellular immunoglobulin domains (2 NH2-terminal, membrane-distal variable-(V)-type (V1, V2 or D1, D2) and 3 membrane-proximal constant-(C2)-type Ig folds) [C1, C2, C3], a transmembrane region, and a short cytoplasmic tail. The N-terminal domain (D1) is exclusively involved in ligand binding, whereas membrane proximal domains (C2, C3 or D4, D5) are required for homophilic interactions.

ALCAM binds to domain 3 of CD6 corresponding to the membrane proximal SRCR domain [11].

Studies of the role of CD6/ALCAM interactions in T-cell regulation have shown that this receptor-ligand pair is able to mediate the adhesion of CD6 expressing cells to thymic epithelial cells [10]. This and other evidence suggests that CD6/ALCAM interactions are important for modulating T-cell development and activation.

Moreover, ALCAM shedding has also been reported, and, like with sCD6, the process appears to be the product of ADAM family metalloproteinase-mediated cleavage. [17] Further, elevated levels of shed ALCAM has been reported in the urine of patients with bladder cancer, where it may serve as a prognostic biomarker of survival. [18] Moreover, in one report, elevated urinary ALCAM and VCAM were observed in SLE patients as compared to healthy patients, but ALCAM was not as highly elevated in active renal samples as compared to healthy samples, leading the investigators to conclude VCAM was a better marker. [19]

Although the functional characterization of CD6 remains incomplete, an anti-CD6 mAb has been successfully applied in a clinical setting to purge bone marrow of T-cells and T-cell precursors. These findings further support the hypothesis that CD6 plays an important role in modulating T-cell function in vivo. CD6 is also reported to be part of the immunologic synapse mediating early and late T-cell-antigen presenting cells (APC) interaction. [12]

U.S. Pat. No. 6,372,215 discloses antibodies and other binding agents that bind specifically to SRCR domains 3 (D3) of human CD6 (hCD6) or human CD6 stalk domain (CD6S) and inhibit activated leukocyte cell adhesion molecule (ALCAM) binding to CD6.

Earlier publications and patents disclosed sequences of the murine anti-CD6 (IOR-T1) monoclonal antibody and the amino acid modifications that were carried out to humanize IOR-T1 to T1h (humanized IOR-T1). U.S. Pat. No. 5,712,120 and its equivalent EP 0699755 disclose specific methods to humanize murine monoclonal antibodies and the sequence of IOR-T1 and T1h. U.S. Pat. No. 6,572,857 and its equivalent EP 0807125 disclose the sequence of IOR-T1 and T1h (humanized IOR-T1). PCT/IN2008/00562, entitled “A Monoclonal Antibody and a Method Thereof,” discloses the production of an anti-CD6 antibody in NS0 cells, which has the heavy and light chain sequences provided herein as SEQ ID NOS: 1 and 2. The INN name for this antibody is itolizumab. Itolizumab is produced in the mouse derived NS0 cell line and in Chinese Hamster Ovary (CHO) cells, and is referred to herein by its trade name EQ001, when produced in CHO cells and by its trade name ALZUMAb, when produced in NS0 cells. EQ001 (i.e., itolizumab produced in CHO cells) is also known in the art as “Bmab-600.” In various embodiments herein, we refer to the antibody itself, irrespective of its production method, by its INN name, itolizumab. Thus, the term itolizumab, as used herein, encompasses ALZUMAb and EQ001, each of which have the same sequence as itolizumab. The amino acid sequences of the variable heavy (VH) and variable light (VK) of itolizumab (and EQ001/ALZUMAb) are provided herein as SEQ ID NOS: 1 and 2, respectively. The nucleotide (DNA) sequences of the VH and VK of itolizumab (and EQ001/ALZUMAb) are provided herein as SEQ ID NOS: 3 and 4, respectively. The amino acid sequence of the itolizumab (and EQ001/ALZUMAb) VH CDRs 1-3 are provided as SEQ ID NOS: 5-7, respectively. The amino acid sequence of the itolizumab (and EQ001/ALZUMAb) VK CDRs 1-3 are provided as SEQ ID NOS: 8-10, respectively.

Antibodies targeting CD6 have shown promise as therapies for a wide-range of diseases and conditions that are caused, at least in part, by aberrant T cell activity. For example, PCT/IN2008/000562 discloses the use of itolizumab to inhibit the proliferation of naïve T cells and to treat various inflammatory disorders including multiple sclerosis, transplant rejection, rheumatoid arthritis, and psoriasis. Indeed ALZUMAb is currently marketed in India for the treatment of psoriasis. Further, the use of itolizumab to treat lupus is disclosed in PCT/IB2017/056428. However, due to the heterogeneity of these diseases and their tendency to cycle between difference disease forms mediated by T cells, B cells, dendritic cells, monocytes, and neutrophils, more targeted treatment therapies are needed to more fully tap the potential of these antibodies.

As of yet, no biomarker strategy is employed clinically to determine when a patient might be most likely to respond favorably to treatment with an anti-CD6 antibody (e.g., EQ001) or more generally to an inhibitor of the CD6-ALCAM pathway.

Accordingly, some aspects of the present disclosure provide a method for identifying whether a subject (or “patient” interchangeably throughout) has an inflammatory or autoimmune disease that is sensitive to CD6-ALCAM pathway inhibition, the method comprising determining whether the subject exhibits an elevated level of soluble CD6 protein, soluble ALCAM protein, or both (e.g., elevated levels in a biological sample obtained from the subject, such as, e.g., a urine sample). In some embodiments, the inflammatory or autoimmune disease is selected from a neuroinflammatory disease, an inflammatory bowel disease, an inflammatory lung disease, an inflammatory kidney disease, and a systemic inflammatory disease. In some embodiments, the inflammatory or autoimmune disease is an inflammatory kidney disease. In some embodiments, the inflammatory kidney disease is selected from LN, IgA nephropathy, anti-neutrophil cytoplasmic antibody-associated glomerulonephritis, autoimmune (formerly idiopathic) membranous nephropathy, anti-glomerular basement membrane glomerulonephritis, and C3 nephropathy. In particular embodiments, the present disclosure provides such a method, wherein the method identifies whether a subject has a form of LN that is sensitive to CD6-ALCAM pathway inhibition based on the levels of soluble CD6 protein, soluble ALCAM protein, or both in a sample obtained from the subject (e.g., from the subject's urine). LN subjects (or SLE or inflammatory or autoimmune disease subject, more generally) found to have elevated levels (e.g., in their urine) of soluble CD6 protein, soluble ALCAM protein, or both may be more likely to have active T cell-driven disease. Moreover, by analyzing the basal concentrations of these markers in a sample (e.g., urine) from a subject known to have inactive LN (or inactive SLE or inflammatory or autoimmune disease subject, more generally), and then subsequently analyzing whether any changes in the levels of these markers occurs over time (e.g., over the course of days, months, years) early detection of an increase in these markers may signal the initial transitional phase of the disease from inactive to active form. This will enable clinicians to administer T cell blocking therapies (and/or other therapies, e.g., immunosuppressives) that may arrest the disease progression in its early phase of reactivation; thus, preventing full-blown transition into active disease state. As is discussed further below, the CD6-ALCAM pathway inhibitor may be any agent capable of blocking or decreasing signaling through the CD6-ALCAM pathway. Such inhibitors include without limitation, anti-CD6 antibodies and anti-ALCAM antibodies, as well as antigen biding fragments thereof. In particular embodiments, the CD6-ALCAM pathway inhibitor is itolizumab. In certain particular embodiments, the CD6-ALCAM pathway inhibitor is EQ001.

Some aspects of the present disclosure provide a method for treating an inflammatory or autoimmune disease with a CD6-ALCAM pathway inhibitor, the method comprising: determining whether a biological sample obtained from a subject having or suspected of having inflammatory or autoimmune disease contains an elevated level of soluble CD6 and/or ALCAM protein; and administering to the subject a CD6-ALCAM pathway inhibitor if the biological sample contains an elevated level of soluble CD6 and/or ALCAM protein. In some embodiments, the inflammatory or autoimmune disease is selected from a neuroinflammatory disease, an inflammatory bowel disease, an inflammatory lung disease, an inflammatory kidney disease, and a systemic inflammatory disease. In some embodiments, the inflammatory or autoimmune disease is an inflammatory kidney disease. In some embodiments, the inflammatory or autoimmune disease is selected from lupus nephritis, IgA nephropathy, anti-neutrophil cytoplasmic antibody-associated glomerulonephritis, autoimmune (formerly idiopathic) membranous nephropathy, anti-glomerular basement membrane glomerulonephritis, and C3 nephropathy. In particular embodiments, the present disclosure provides such a method, wherein the method is for treating LN with the CD6-ALCAM pathway inhibitor, the method comprising: determining whether a biological sample obtained from a subject having or suspected of having LN contains an elevated level of soluble CD6 and/or ALCAM protein; and administering to the subject a CD6-ALCAM pathway inhibitor if the biological sample contains an elevated level of soluble CD6 and/or ALCAM protein. As is discussed further below, the CD6-ALCAM pathway inhibitor may be any agent capable of blocking or decreasing signaling through the CD6-ALCAM pathway. Such inhibitors include without limitation, anti-CD6 antibodies and anti-ALCAM antibodies, as well as antigen biding fragments thereof. In particular embodiments, the CD6-ALCAM pathway inhibitor is itolizumab. In certain particular embodiments, the CD6-ALCAM pathway inhibitor is EQ001.

Some aspects of the present disclosure provide a method for using a CD6-ALCAM pathway inhibitor to treat a subject with an inflammatory or autoimmune disease, the method comprising the steps of: determining whether the subject exhibits elevated soluble CD6 and/or ALCAM protein; and administering to the subject the CD6-ALCAM pathway inhibitor if the subject exhibits elevated soluble CD6 and/or ALCAM protein. In some embodiments, the inflammatory or autoimmune disease is selected from a neuroinflammatory disease, an inflammatory bowel disease, an inflammatory lung disease, an inflammatory kidney disease, and a systemic inflammatory disease. In some embodiments, the inflammatory or autoimmune disease is an inflammatory kidney disease. In some embodiments, the inflammatory or autoimmune disease is selected from lupus nephritis, IgA nephropathy, anti-neutrophil cytoplasmic antibody-associated glomerulonephritis, autoimmune (formerly idiopathic) membranous nephropathy, anti-glomerular basement membrane glomerulonephritis, and C3 nephropathy. In particular embodiments, the present disclosure provides such a method for using a CD6-ALCAM pathway to treat a subject with lupus nephritis, the method comprising the steps of: determining whether the subject exhibits elevated levels of soluble CD6 and/or ALCAM protein; and administering to the subject the CD6-ALCAM pathway inhibitor if the subject exhibits elevated levels of soluble CD6 and/or ALCAM protein. As is discussed further below, the CD6-ALCAM pathway inhibitor may be any agent capable of blocking or decreasing signaling through the CD6-ALCAM pathway. Such inhibitors include without limitation, anti-CD6 antibodies and anti-ALCAM antibodies, as well as antigen biding fragments thereof. In particular embodiments, the CD6-ALCAM pathway inhibitor is itolizumab. In certain particular embodiments, the CD6-ALCAM pathway inhibitor is EQ001.

Some aspects of the present disclosure provide a method for treating a subject with a CD6-ALCAM pathway inhibitor, wherein the subject has an inflammatory or autoimmune disease, the method comprising the steps of: (A) determining whether the subject has a CD6-ALCAM pathway inhibitor-sensitive disease by: (i) obtaining or having obtained a biological sample from the subject; and (ii) performing or having performed an assay on the biological sample to determine if the sample exhibits an elevated level of soluble CD6 and/or ALCAM protein; and (B) administering the CD6-ALCAM pathway inhibitor to the subject if the subject has elevated soluble CD6 and/or ALCAM protein. In some embodiments, the inflammatory or autoimmune disease is selected from a neuroinflammatory disease, an inflammatory bowel disease, an inflammatory lung disease, an inflammatory kidney disease, and a systemic inflammatory disease. In some embodiments, the inflammatory or autoimmune disease is an inflammatory kidney disease. In some embodiments, the inflammatory or autoimmune disease is selected from lupus nephritis, IgA nephropathy, anti-neutrophil cytoplasmic antibody-associated glomerulonephritis, autoimmune (formerly idiopathic) membranous nephropathy, anti-glomerular basement membrane glomerulonephritis, and C3 nephropathy. In particular embodiments, the present disclosure provides such a method for treating a subject with a CD6-ALCAM pathway inhibitor, wherein the subject has lupus nephritis, the method comprising the steps of: (A) determining whether the subject has a CD6-ALCAM pathway inhibitor-sensitive disease by: (i) obtaining or having obtained a biological sample from the subject; and (ii) performing or having performed an assay on the biological sample to determine if the sample exhibits an elevated level of soluble CD6 and/or ALCAM protein; and (B) administering the CD6-ALCAM pathway inhibitor to the subject if the subject has elevated soluble CD6 and/or ALCAM protein. As is discussed further below, the CD6-ALCAM pathway inhibitor may be any agent capable of blocking or decreasing signaling through the CD6-ALCAM pathway. Such inhibitors include without limitation, anti-CD6 antibodies and anti-ALCAM antibodies, as well as antigen biding fragments thereof. In particular embodiments, the CD6-ALCAM pathway inhibitor is itolizumab. In certain particular embodiments, the CD6-ALCAM pathway inhibitor is EQ001.

Some aspects of the present disclosure provide a method of predicting the prognosis of a subject with an inflammatory or autoimmune disease, the method comprising the steps of: (i) obtaining or having obtained a plurality of biological samples from the subject over a time course; and (ii) performing or having performed an assay on each of the biological samples to determine if there is a change over time in the level of soluble CD6 and/or ALCAM proteins that are present in the sample; wherein (a) if the sample exhibits an increase in the level of soluble CD6 and/or ALCAM protein over time, then the prognosis is determined to be poor; (b) if the sample exhibits no change in the level of soluble CD6 and/or ALCAM protein over time, then the prognosis is determined to be neutral; and (c) if the sample exhibits a decrease in the level of soluble CD6 and/or ALCAM protein over time, then the prognosis is determined to be good. The time course may be any suitable time course. In some embodiments, the time course is performed over a course of days, weeks, months or years. In some embodiments, the inflammatory or autoimmune disease is selected from a neuroinflammatory disease, an inflammatory bowel disease, an inflammatory lung disease, an inflammatory kidney disease, and a systemic inflammatory disease. In some embodiments, the inflammatory or autoimmune disease is an inflammatory kidney disease. In some embodiments, the inflammatory or autoimmune disease is selected from lupus nephritis, IgA nephropathy, anti-neutrophil cytoplasmic antibody-associated glomerulonephritis, autoimmune (formerly idiopathic) membranous nephropathy, anti-glomerular basement membrane glomerulonephritis, and C3 nephropathy. In particular embodiments, the present disclosure provides such a method for predicting the prognosis of a subject with LN, the method comprising the steps of: obtaining or having obtained a plurality of biological samples from the subject over a time course; and (ii) performing or having performed an assay on each of the biological samples to determine if there is a change over time in the level of soluble CD6 and/or ALCAM proteins that are present in the sample; wherein (a) if the sample exhibits an increase in the level of soluble CD6 and/or ALCAM protein over time, then the prognosis is determined to be poor; (b) if the sample exhibits no change in the level of soluble CD6 and/or ALCAM protein over time, then the prognosis is determined to be neutral; and (c) if the sample exhibits a decrease in the level of soluble CD6 and/or ALCAM protein over time, then the prognosis is determined to be good. The time course may be any suitable time course. In some embodiments, the time course is performed over a course of days, weeks, months or years. Such prognostic methods may further comprise administering to the subject a CD6-ALCAM pathway inhibitor. As is discussed further below, the CD6-ALCAM pathway inhibitor may be any agent capable of blocking or decreasing signaling through the CD6-ALCAM pathway. Such inhibitors include without limitation, anti-CD6 antibodies and anti-ALCAM antibodies, as well as antigen biding fragments thereof. In particular embodiments, the CD6-ALCAM pathway inhibitor is itolizumab. In certain particular embodiments, the CD6-ALCAM pathway inhibitor is EQ001.

Some aspects of the present disclosure provide a method of determining whether a subject has active inflammatory or autoimmune disease comprising (a) determining a first concentration of soluble CD6 and/or ALCAM protein present in a sample from the subject; (b) determining a second concentration, or average concentration, of soluble CD6 and/or ALCAM protein present in a similar sample from a control person, or a population of control persons, respectively, that do not have active inflammatory or autoimmune disease; and (c) determining that the subject has active inflammatory or autoimmune disease if the first concentration is greater than the second concentration. In some embodiments, the inflammatory or autoimmune disease is selected from a neuroinflammatory disease, an inflammatory bowel disease, an inflammatory lung disease, an inflammatory kidney disease, and a systemic inflammatory disease. In some embodiments, the inflammatory or autoimmune disease is an inflammatory kidney disease. In some embodiments, the inflammatory or autoimmune disease is selected from lupus nephritis, IgA nephropathy, anti-neutrophil cytoplasmic antibody-associated glomerulonephritis, autoimmune (formerly idiopathic) membranous nephropathy, anti-glomerular basement membrane glomerulonephritis, and C3 nephropathy. In particular embodiments, the present disclosure provides such a method for determining whether a subject has active lupus nephritis comprising (a) determining a first concentration of soluble CD6 and/or ALCAM protein present in a sample from the subject; (b) determining a second concentration, or average concentration, of soluble CD6 and/or ALCAM protein present in a similar sample from a control person, or a population of control persons, respectively, that do not have active lupus nephritis; and (c) determining that the subject has active nephritis if the first concentration is greater than the second concentration. In some instances, the sample that is analyzed is a urine sample. In some instances the sample that is analyzed is a urine sample when the disease is an inflammatory kidney disease (e.g., LN). methods of determining Such methods of determining whether a subject has active inflammatory or autoimmune disease (e.g., active LN) may further comprise administering to the subject a CD6-ALCAM pathway inhibitor if the patient is determined by the method to have active inflammatory or autoimmune disease (e.g., active LN). As is discussed further below, the CD6-ALCAM pathway inhibitor may be any agent capable of blocking or decreasing signaling through the CD6-ALCAM pathway. Such inhibitors include without limitation, anti-CD6 antibodies and anti-ALCAM antibodies, as well as antigen biding fragments thereof. In particular embodiments, the CD6-ALCAM pathway inhibitor is itolizumab. In certain particular embodiments, the CD6-ALCAM pathway inhibitor is EQ001.

Some aspects of the present disclosure provide a method of determining whether a subject has transitioned from inactive inflammatory or autoimmune disease to active inflammatory or autoimmune disease comprising (a) determining a first concentration of soluble CD6 and/or ALCAM protein present in a first sample from the subject; wherein the first sample is obtained from the subject when the subject has inactive lupus nephritis; (b) determining a second concentration of soluble CD6 and/or ALCAM protein present in one or more second samples from the subject; wherein each second sample is obtained from the subject after the first sample was obtained; and (c) determining that the subject has active lupus nephritis or is transitioning into active nephritis if the second concentration of soluble CD6 and/or ALCAM protein is greater than the first concentration. In some embodiments, the inflammatory or autoimmune disease is selected from a neuroinflammatory disease, an inflammatory bowel disease, an inflammatory lung disease, an inflammatory kidney disease, and a systemic inflammatory disease. In some embodiments, the inflammatory or autoimmune disease is an inflammatory kidney disease. In some embodiments, the inflammatory or autoimmune disease is selected from lupus nephritis, IgA nephropathy, anti-neutrophil cytoplasmic antibody-associated glomerulonephritis, autoimmune (formerly idiopathic) membranous nephropathy, anti-glomerular basement membrane glomerulonephritis, and C3 nephropathy. In particular embodiments, the present disclosure provides such a method for determining whether a subject has transitioned from inactive lupus nephritis to active lupus nephritis comprising (a) determining a first concentration of soluble CD6 and/or ALCAM protein present in a first sample from the subject; wherein the first sample is obtained from the subject when the subject has inactive lupus nephritis; (b) determining a second concentration of soluble CD6 and/or ALCAM protein present in one or more second samples from the subject; wherein each second sample is obtained from the subject after the first sample was obtained; and (c) determining that the subject has active lupus nephritis or is transitioning into active nephritis if the second concentration of soluble CD6 and/or ALCAM protein is greater than the first concentration. Such methods of determining whether a subject has transitioned from inactive inflammatory or autoimmune disease to active inflammatory or autoimmune disease (e.g., transitioned from inactive LN to active LN) may further comprise administering to the subject a CD6-ALCAM pathway inhibitor. As is discussed further below, the CD6-ALCAM pathway inhibitor may be any agent capable of blocking or decreasing signaling through the CD6-ALCAM pathway. Such inhibitors include without limitation, anti-CD6 antibodies and anti-ALCAM antibodies, as well as antigen biding fragments thereof. In particular embodiments, the CD6-ALCAM pathway inhibitor is itolizumab. In certain particular embodiments, the CD6-ALCAM pathway inhibitor is EQ001.

### Inflammatory or Autoimmune Diseases

As will be apparent to one skilled in the all of the method disclosed herein may in some embodiments be employed in connection with any inflammatory or autoimmune disease. In some embodiments, the inflammatory or autoimmune disease is selected from a neuroinflammatory diseases, an inflammatory bowel diseases, an inflammatory lung diseases, an inflammatory kidney diseases, and a systemic inflammatory diseases. In some embodiments, the inflammatory or autoimmune disease is an inflammatory kidney disease. In some embodiments, the inflammatory or autoimmune disease is selected from lupus nephritis, IgA nephropathy, anti-neutrophil cytoplasmic antibody-associated glomerulonephritis, autoimmune (formerly idiopathic) membranous nephropathy, anti-glomerular basement membrane glomerulonephritis, and C3 nephropathy.

### Samples

In some embodiments, any one of the methods disclosed herein may be performed on or utilize a sample obtained from a subject that has or is suspected of having an inflammatory or autoimmune disease. For example, in some embodiments, the sample may be obtained from a subject that has or is suspected of having a neuroinflammatory disease, an inflammatory bowel disease, an inflammatory lung disease, an inflammatory kidney disease, or a systemic inflammatory diseases. The sample may be obtained from a subject that has or is suspected of having lupus. The sample may be obtained from a subject that has or is suspected of having SLE. The sample may be obtained from a subject that has or is suspected of having an inflammatory kidney disease. The sample may be obtained from a subject that has or is suspected of having an inflammatory kidney disease selected from IgA nephropathy, anti-neutrophil cytoplasmic antibody-associated glomerulonephritis, autoimmune (formerly idiopathic) membranous nephropathy, anti-glomerular basement membrane glomerulonephritis, and C3 nephropathy. The sample may be obtained from a subject that has or is suspected of having LN.

The samples utilized in the methods disclosed herein may be from any suitable source. For example, the sample may be a biopsy sample (e.g., a liver biopsy sample). In some preferred instances, the sample may be obtainable via a non-invasive or minimally invasive procedure. For example, in some instances the sample may be selected from any bodily fluid. In some instances the sample may be selected from any one of blood, serum, urine, sputum, Cerebrospinal fluid (CSF), Bronchoalveolar lavage fluid (BALF), and stool. In certain preferred embodiments, the sample is urine (e.g., urine from a subject that has or is suspected of having LN).

In certain embodiments, the optimal sample source is determined by the type of inflammatory or autoimmune disease that the subject has or is suspected of having. For example, in certain embodiments, the inflammatory or autoimmune disease is an iinflammatory kidney disease and the sample is urine. In certain embodiments, the inflammatory or autoimmune disease is a neuroinflammatory disease and the sample is CSF. In certain embodiments, the inflammatory or autoimmune disease is an inflammatory bowel disease and the sample is stool. In certain embodiments, the inflammatory or autoimmune disease is an inflammatory lung disease and the sample is sputum or BALF. In certain embodiments, the inflammatory or autoimmune disease is a systemic inflammatory disease and the sample is blood or serum.

In some instances, it may be useful to collect multiple samples from a single subject, e.g., to monitor the subject's levels of soluble CD6 and/or soluble ALCAM over the course of time. In such instances, the time separating the collection of the samples is not rigidly defined, and the best course may be determined by a clinician in accordance with well-known procedures. For example, samples may be collected over a time course of days, weeks, months, or years. Decreases or increases in the observed levels of soluble CD6 and/or soluble ALCAM in the samples over the time course may inform on progression of the disease (e.g., an inflammatory or autoimmune disease such as, e.g., LN) from an active to inactive state, or from an inactive to active state. Changes in the observed levels of soluble CD6 and/or soluble ALCAM in the samples over the time course may inform on prognosis of the patient's disease. Moreover, changes in the observed levels of soluble CD6 and/or soluble ALCAM in the samples over the time course may be monitored before and after administration of a therapeutic agent, e.g., a CD6-ALCAM pathway inhibitor disclosed herein or a steroid or immunosuppressant, in order to determine whether any effect of the therapeutic agent on the activity of the inflammatory or autoimmune disease (e.g., LN) is observed. For example, changes in the observed levels of soluble CD6 and/or soluble ALCAM in the samples over the time course may be monitored before and after administration of any agent capable of blocking or decreasing signaling through the CD6-ALCAM pathway. In some instances, changes in the observed levels of soluble CD6 and/or soluble ALCAM in the samples over the time course may be monitored before and after administration of a CD6-ALCAM pathway inhibitor selected from an anti-CD6 antibody an anti-ALCAM antibody, as well as antigen biding fragments of such antibodies, or a combination thereof. In particular embodiments, changes in the observed levels of soluble CD6 and/or soluble ALCAM in the samples over the time course may be monitored before and after administration of itolizumab. In certain particular embodiments, changes in the observed levels of soluble CD6 and/or soluble ALCAM in the samples over the time course may be monitored before and after administration of EQ001. In some embodiments, the administration of the CD6-ALCAM pathway inhibitor and/or a steroid or immunosuppressant results in a decrease in the detected levels of soluble CD6 and/or soluble ALCAM in the subject's sample over the time course. In some embodiments, such a decrease signals that the therapy is effective and the active disease (e.g., active inflammatory or autoimmune disease such as, e.g., active LN) is transitioning to an inactive state or has transitioned to an inactive state.

### Detection Methods

The soluble CD6 protein detected in the methods disclosed herein may be a full length CD6 protein or a fragment of a CD6 protein. The fragment of the CD6 protein may be an extracellular portion of a CD6 protein, or a fragment thereof. Any detectable portion of soluble CD6 may be targeted for detection in accordance with the presently disclosed methods.

The soluble ALCAM protein detected in the methods disclosed herein may be a full length ALCAM protein or a fragment of an ALCAM protein. The fragment of the full length ALCAM protein may be an extracellular portion of an ALCAM protein, or a fragment thereof. Any detectable portion of soluble ALCAM may be targeted for detection in accordance with the presently disclosed methods.

The soluble CD6 protein or soluble ALCAM protein detected in the methods disclosed herein may detected by any means known in the art or disclosed herein. Numerous protein detection methods are known in the art and are suitable for use in the present methods. For example, but not to be limited in any way, the soluble CD6 protein or soluble ALCAM protein detected by single-plex ELISA; multiplex ELISA, bead-based immunocapture with FACs-based detection; bead-based immunocapture with ELISA-based detection; bead-based immunocapture with chemiluminescent-based detection; meso-scale diagnostic (MSD); western blot, quantitative western blot; high performance liquid chromatography (HPLC); mass spectrometry; and a combination thereof. Such methods are known in the art.

The detection of the soluble CD6 protein or soluble ALCAM protein may be qualitative.

The detection of the soluble CD6 protein or soluble ALCAM protein may be quantitative. Quantitative detection may include comparison of the detected levels of soluble CD6 protein or soluble ALCAM protein to a known quantity of soluble CD6 protein or soluble ALCAM protein, respectively. Such a comparison may utilize a standard curve. The creation of standard curves and the use of such curves to quantify the amount of protein in an unknown sample is routine in the art and such methods will be apparent to the skilled artisan and may include, without limitation, comparison of detected levels of unknown concentrations of soluble CD6 protein or soluble ALCAM protein in a sample to detected levels of a serial dilution of standard control samples of soluble CD6 protein or soluble ALCAM protein (or fragments thereof) of known concentrations.

In some embodiments, the detection of soluble CD6 and/or soluble ALCAM further comprises determining a measured concentration value for the soluble CD6 and/or soluble ALCAM in a sample(s) and comparing the measured value to a threshold value, wherein either an “active” or “inactive” level of the soluble CD6 and/or soluble ALCAM is identified. In some embodiments, the method assigns a likelihood, risk, or probability that such that an event of interest is more or less likely to occur within 180 Days of the time at which the body fluid sample is obtained from the subject. In some embodiments, the assigned likelihood, risk, or probability relates to an event of interest occurring within a time period including, but not limited to, 18 months, 120 Days, 90 Days, 60 Days, 45 Days, 30 Days, 21 Days, 14 Days, 7 Days, 5 Days, 96 hours, 72 hours, 48 hours, 36 hours, 24 hours, 12 hours, or less. Alternatively, assigning a risk at 0 hours of the time at which the body fluid sample is obtained from the subject is equivalent to diagnosis of a current condition (e.g., active or inactive inflammatory or autoimmune disease).

Selecting a diagnostic threshold involves, among other things, consideration of the probability of disease, distribution of true and false diagnoses at different test thresholds, and estimates of the consequences of treatment (or a failure to treat) based on the diagnosis. For example, when considering administering a specific CD6-ALCAM pathway inhibitor (e.g., EQ001) which is highly efficacious and has a low level of risk, few tests are needed because clinicians can accept substantial diagnostic uncertainty. On the other hand, in situations where treatment options are less effective and more risky, clinicians often need a higher degree of diagnostic certainty. Thus, a cost/benefit analysis is involved in selecting a diagnostic threshold.

In some embodiments, the present invention provides for the detection of a CD6 and/or ALCAM polynucleotide in a biological sample (e.g., urine). For example, the methods may in some embodiments involve detection of mRNA expression of CD6 and/or ALCAM.

The detection of the levels of a polynucleotide in the sample can be carried out by any of the methods known in the state of the art. For example, the detection method may involve hybridization of the nucleic acids by contact between a probe and the target CD6 or ALCAM nucleic acid under conditions where the probe and its complementary target can form stable hybrid duplexes by pairing complementary bases. Nucleic acid hybridization methods are well known in the art. The probe may be labeled with a fluorescent molecule. Hybridized nucleic acids are detected by detecting one or more labels of the sample nucleic acids and probes. Labels can be incorporated by any of the methods known to those skilled in the art. Commonly used label tags include, but are not limited to, biotin, fluorescent molecules, radioactive molecules, chromogenic substrates, chemiluminescent markers, enzymes, and the like. The methods for biotinylation nucleic acids are well known in the art, as are methods for introducing fluorescent molecules and radioactive molecules into oligonucleotides and nucleotides.

ALCAM and CD6 mRNA levels can be determined by reverse-transcription (RT) PCR and by quantitative RT-PCR (QRT-PCR) or real-time PCR methods. Methods of RT-PCR and QRT-PCR are well known in the art.

In some embodiments, the level of a CD6 or ALCAM mRNA can be measured by a quantitative sequencing technology, e.g. a quantitative next-generation sequence technology. Methods of sequencing a nucleic acid sequence are well known in the art. Briefly, a sample obtained from a subject can be contacted with one or more primers which specifically hybridize to a single-strand nucleic acid sequence flanking the target gene sequence and a complementary strand is synthesized. In some next-generation technologies, an adaptor (double or single-stranded) is ligated to nucleic acid molecules in the sample and synthesis proceeds from the adaptor or adaptor compatible primers. In some related technologies, the sequence can be determined, e.g. by determining the location and pattern of the hybridization of probes, or measuring one or more characteristics of a single molecule as it passes through a sensor (e.g. the modulation of an electrical field as a nucleic acid molecule passes through a nanopore). Exemplary methods of sequencing include, but are not limited to, Sanger sequencing, dideoxy chain termination, 454 sequencing, SOLiD sequencing, polony sequencing, Illumina sequencing, Ion Torrent sequencing, sequencing by hybridization, nanopore sequencing, Helioscope sequencing, single molecule real time sequencing, RNAP sequencing, and the like. Methods and protocols for performing these sequencing methods are known in the art, see, e.g. “Next Generation Genome Sequencing” Ed. Michal Janitz, Wiley-VCH; “High-Throughput Next Generation Sequencing” Eds. Kwon and Ricke, Humanna Press, 2011; and Sambrook et al., Molecular Cloning: A Laboratory Manual (4 ed.), Cold Spring Harbor Laboratory Press, Cold Spring Harbor, N.Y., USA (2012); which are incorporated by reference herein in their entireties.

In particular embodiments, CD6 or ALCAM mRNA (e.g., mRNA present in a cell isolated from a urine sample from a person known or suspected to have LN) may be detected by RNA-Sequencing (RNA-Seq). RNA Sequencing. RNA-seq (RNA Sequencing), also called Whole Transcriptome Shotgun Sequencing (WTSS), is a technology that is well known in the art. It utilizes the capabilities of Next-Generation Sequencing (NGS) to reveal a snapshot of RNA presence and quantity from a genome at a given moment in time. The transcriptome of a cell is dynamic; it continually changes as opposed to a static genome. The recent developments of next-generation sequencing allow for increased base coverage of a DNA sequence, as well as higher sample throughput. This facilitates sequencing of the RNA transcripts in a cell, providing the ability to look at alternative gene spliced transcripts, post-transcriptional changes, gene fusion, mutations/SNPs and changes in gene expression. In addition to mRNA transcripts, RNA-Seq can look at different populations of RNA to include total RNA, small RNA, such as miRNA, tRNA, and ribosomal profiling. RNA-Seq can also be used to determine exon/intron boundaries and verify or amend previously annotated 5′ and 3′ gene boundaries. Prior to NGS, transcriptomics and gene expression studies were previously done with expression microarrays, which contain thousands of DNA sequences that probe for a match in the target sequence, making available a profile of all transcripts being expressed. Such microarrays may also be used to detect CD6 or ALCAM mRNA express, as may be Serial Analysis of Gene Expression (SAGE), each of which technologies are well-known in the art.

Polynucleotide and ribonucleic acid (RNA) molecules can be isolated from a particular biological sample (e.g., a kidney biopsy or cells obtained from a urine sample) using any of a number of procedures, which are well-known in the art, the particular isolation procedure chosen being appropriate for the particular biological sample. For example, freeze-thaw and alkaline lysis procedures can be useful for obtaining nucleic acid molecules from solid materials; heat and alkaline lysis procedures can be useful for obtaining nucleic acid molecules from urine: and proteinase K extraction can be used to obtain nucleic acid from blood (Roiff, A et al. PCR: Clinical Diagnostics and Research, Springer (1994)).

### Thresholds

Suitable thresholds may be determined in a variety of ways. For example, one recommended diagnostic threshold for the diagnosis of active LN may set a diagnostic threshold at the 97.5th percentile of the soluble CD6 and/or soluble ALCAM concentration measured in a normal population. Another method to determine a diagnostic threshold may comprise measuring serial samples from the same patient, where a prior “baseline” result is used to monitor for temporal changes in a biomarker level. Population studies may also be used to select thresholds. For example, Receiver Operating Characteristic (“ROC”) analysis is often used to select a threshold to distinguish a “diseased” subpopulation from a “non-diseased” subpopulation. Predictive power balances the occurrences of false positives (i.e., for example, when the person tests positive, but actually does not have the disease) and false negatives (i.e., for example, when the person tests negative, suggesting they are healthy, when they actually do have the disease). To draw a ROC curve, the true positive rate (TPR) and false positive rate (FPR) are determined as the decision threshold is varied continuously. Since TPR is equivalent with sensitivity and FPR is equal to (1—specificity), the ROC graph is sometimes called the sensitivity vs (1—specificity) plot. A perfect test will have an area under the ROC curve of 1.0; a random test will have an area of 0.5. A threshold value is selected to provide an acceptable level of specificity and sensitivity usually determined by summing specificity values with sensitivity values. Consequently, the larger the calculated threshold value the greater the predictive power of the specific assay measurement under analysis.

In this context, “diseased” is meant to refer to a population having one characteristic (e.g., the presence of an active inflammatory or autoimmune disease or condition or the occurrence of some outcome) and “non-diseased” population lacking the same characteristic (e.g., the presence of an inactive inflammatory or autoimmune disease or condition).

While a single decision threshold is the simplest application of such a method, multiple decision thresholds may be used. For example, below a first threshold, the absence of disease may be assigned with relatively high confidence, and above a second threshold the presence of disease may also be assigned with relatively high confidence. Between the two thresholds may be considered indeterminate. This is meant to be exemplary in nature only.

In addition to threshold value comparisons, other methods for correlating assay measurements to a patient classification (e.g., occurrence or nonoccurrence of disease, likelihood of an outcome, etc.) include, but are not limited to, decision trees, rule sets, Bayesian methods, and neural network methods. These methods can produce probability values representing the degree to which a subject or patient belongs to one classification out of a plurality of classifications.

### CD6-ALCAM Pathway Inhibitors

In some embodiments, any agent capable of inhibiting the CD6-ALCAM pathway is suitable for use as the CD6-ALCAM pathway inhibitor utilized in the methods disclosed herein.

In certain aspects, the CD6-ALCAM pathway inhibitor is an anti-CD6 antibody. Anti-CD6 antibodies are known in the art, and disclosed herein. Any one or more of the anti-CD6 antibodies disclosed herein may be used in any one of the methods disclosed herein. For example, in certain preferred instances, the anti-CD6 antibody is EQ001.

In certain aspects, the anti-CD6 antibody may be any antibody that binds to CD6 and blocks CD6-mediated downstream signaling in a T cell. For example, blocking studies using anti-CD6 monoclonal antibodies (mAbs) suggest that CD6 plays an important role in T cell development by regulating T cell adhesive interactions with thymic epithelial (TE) cells (Patel et al., J. Exp. Med. (1995) 181:1563-1568). Additional studies have shown that CD6 can function as an important accessory molecule in T cell activation. For example, certain anti-CD6 mAb are directly mitogenic for T cells (Gangemi et al., J. Immunol. (1989) 143:2439; Bott et al., Int. Immunol. (1993) 7:783), whereas others are able to co-stimulate T cell proliferation in conjunction with anti-CD3, anti-CD2 or PMA (Gangemi et al., J. Immunol. (1989) 143:2439; Morimoto et al., J. Immunol. (1988) 140:2165-2170; Osorio et al., Cell. Immunol. (1994) 154:23). Yet additional evidence of the role of CD6 in T cell activation comes from studies showing that CD6 becomes hyperphosphorylated on Ser and Thr residues (Swack et al., Mol. Immunol. (1989) 26:1037-1049; Swack et al., J. Biol. Chem. (1991) 266:7137; Cardenas et al., J. Immunol., 145:1450-1455 (1990)) and phosphorylated on Tyr residues (Wee et al., J. Exp. Med. (1993) 177:219-223) following T cell activation. These and other studies implicate CD6 as an important modulator of both immature and mature T cell function in vivo, affecting both T cell activation and signal transduction (De Wit, J., et al., Blood (2011) 118:6107-6114), and any antibody that is able to prevent these effects is suitable for use in the present invention.

Accordingly, the anti-CD6 antibody may be an anti-CD6 monoclonal antibody that comprises a heavy chain and light chain variable region comprising an amino acid sequence as set forth in SEQ ID NO: 1 and SEQ ID NO: 2.

The anti-CD6 antibody may be an anti-CD6 monoclonal antibody that comprises a heavy chain and light chain variable region comprising the nucleotide sequence set forth in SEQ ID NO: 3 or a complement thereof, and (b) a nucleic acid molecule comprising the nucleotide sequence set forth in SEQ ID NO: 4 or a complement thereof.

The anti-CD6 antibody may be an anti-CD6 monoclonal antibody that comprises a heavy chain and light chain variable region comprising an amino acid sequence which is at least 80% homologous to the amino acid sequence as set forth in SEQ ID NO: 1 and SEQ ID NO: 2.

The anti-CD6 antibody may be an anti-CD6 monoclonal antibody that specifically binds CD6 and comprises at least about 65% amino acid sequence identity or homology, at least about 70% amino acid sequence identity or homology, at least about 75% amino acid sequence identity or homology, at least about 80% amino acid sequence identity or homology, at least about 80% amino acid sequence identity or homology, at least about 85% amino acid sequence identity or homology, at least about 90% amino acid sequence identity or homology, at least about 95% amino acid sequence identity or homology, at least about 98% amino acid sequence identity or at least about 99% amino acid sequence identity or homology in that portion corresponding to amino acid residues represented by the SEQ ID Nos 1 & 2.

The anti-CD6 antibody may comprise one or more CDRs selected from Itolizumab heavy chain CDR1: GFKFSRYAMS (SEQ ID NO: 5); Itolizumab heavy chain CDR2: TISSGGSYIYYPDSVKG (SEQ ID NO: 6); Itolizumab heavy chain CDR3: RDYDLDYFDS (SEQ ID NO: 7); Itolizumab light chain CDR1: KASRDIRSYLT (SEQ ID NO: 8); Itolizumab light chain CDR2: YATSLAD (SEQ ID NO: 9); Itolizumab light chain CDR3: LQHGESP (SEQ ID NO: 10); and combinations thereof.

In particular embodiments, the anti-CD6 antibody comprises each of the Itolizumab CDRs provided as SEQ ID NOS: 5-10. In particular embodiments, the anti-CD6 antibody is a humanized antibody that comprises each of the Itolizumab CDRs provided as SEQ ID NOS: 5-10. In particular embodiments, the anti-CD6 antibody is a humanized IgG antibody that comprises each of the Itolizumab CDRs provided as SEQ ID NOS: 5-10. In particular embodiments, the anti-CD6 antibody is a humanized IgG1 antibody that comprises each of the Itolizumab CDRs provided as SEQ ID NOS: 5-10. In particular embodiments, the anti-CD6 antibody is a humanized antibody produced in a CHO cell, wherein the humanized antibody comprises each of the Itolizumab CDRs provided as SEQ ID NOS: 5-10.

The anti-CD6 antibody may be selected from UMCD6 mAb (Li et al., PNAS Mar. 7, 2017, vol. 114, no. 10, 2687-2692, incorporated herein by reference in its entirety) and any one of the antibodies listed on Table 1:

TABLE 1

| Anti-CD6 antibodies | | | | |
|---|---|---|---|---|
| Name | Specificity | Isotype | Notes | References |
| OX126 | Human CD6 domain 3 | Mouse IgG1 | Raised against recombinant CD6 domain 3 and cross reacts with | [5] |
| | | | rat CD6. Blocks soluble CD166 binding to CD6. Blocks inter- | |
| | | | actions between cells. | |
| T12 | Human CD6 domain 1 | mouse IgM | Blocks interactions between cells. Used clinically to deplete T. | [12] and reviewed |
| | | | cells. | by [51] |
| UMCD6 | Human CD6 domain 1 | Mouse IgG1 | Highest affinity for CD6 than MT605. Competes with IOR-T1. | [57] |
| | | | Blocks interactions between cells. | |
| MT605 | Human CD6 domain 1 | Mouse IgG1 | Lower affinity for [symbol] CD6 than UMCD6. Partially competes | [57] |
| | | | with IOR-T1. Blocks interactions between cells. | |
| IOR- | Human CD6 domain 1 | Mouse IgG2a/ | Competes with UMCD6 and partially with MT605. Blocks inter- | [57] |
| T1/T1h | | human IgG1 | actions between cells. Used clinically as an immunosuppressant. | |
| 34-81 | Human CD166V2. | Mouse IgG1 | Partially inhibits soluble CD6 binding to cells. Can both promote | [13, 29, 39] |
| | Bound VV but not VI or | | and inhibit CD166 homophilic interactions presumably depending | |
| | VCCC. | | on [symbol] | |
| AZN- | Human CD166 C2C3 | Mouse IgG2a | Inhibits CD166 homophilic [symbol] interactions proposed to be by | [29] |
| L50 | | | inhibiting CD166 hemophilic cis associations. | |
| MAB656 | Human CD166 VCCC | Mouse IgG1 | Inhibitory effects reported in humans and mice but does not bond | [59], Lee J. [symbol] |
| | | | to recombinant mouse CD166. | unpublished data |
| I/F8 | Human CD166 domain 1 | Human single | Blocks CD6/CD166 and CD166/CD166 interactions and blocks | [60] |
| | | chain antibody | [symbol] and [symbol] CD166. | |
| | | fragment | | |

[symbol]

indicates data missing or illegible when filed

The anti-CD6 antibody may be T1h as disclosed in U.S. Pat. No. 8,524,233, incorporated herein by reference in its entirety. The anti-CD6 antibody may be Itolizumab. The anti-CD6 antibody may be ALZUMAb.

The anti-CD6 antibody may be an antibody produced by secreting hybridoma IOR-T1A deposited with the ECACC as deposit No. ECACC 96112640, or a humanize version thereof.

The anti-CD6 antibody may bind to CD6 on the surface of a T cell. The anti-CD6 antibody may bind to domain 1, domain 2, or domain 3 of CD6 on the surface of a T cell. In certain aspects the anti-CD6 antibody binds to domain 1 or domain 3 on CD6. In particular embodiments, the anti-CD6 antibody binds to domain 3 on CD6. The binding of the anti-CD6 antibody to the CD6 on the surface of the T cell may modulate the activity of the T cell. In certain aspects, the binding of the anti-CD6 antibody to CD6 on the surface of a T cell modulates the activity and/or migration of the T cell. In particular aspects, the binding of the anti-CD6 antibody to CD6 on the surface of a T cell modulates migration of the T cell into and through a tissue affected by an inflammatory or autoimmune diseases. Such a tissue may be, e.g., skin, Joints, internal organs, including lung, heart, and kidneys.

The anti-CD6 antibody (e.g., EQ001) may be delivered to the subject as an anti-CD6 pharmaceutical composition.

Pharmaceutical compositions suitable for the delivery of CD6-ALCAM pathway inhibitor (e.g., an anti-CD6 antibody such as EQ001) and methods for their preparation will be readily apparent to those skilled in the art. Such compositions and methods for their preparation may be found, e.g., in Remington's Pharmaceutical Sciences, 19th Edition (Mack Publishing Company, 1995), incorporated herein by reference in its entirety. Pharmaceutical compositions containing anti-CD6 antibodies are also known in the art. For example, the anti-CD6 antibody may be a pharmaceutical composition disclosed in U.S. patent application Ser. No. 12/525,449 (US20100047242), incorporated herein by reference in its entirety.

Pharmaceutical compositions of the present invention may comprise an active pharmaceutical agent such as a CD6-ALCAM pathway inhibitor (e.g., an anti-CD6 antibody such as EQ001) and one or more pharmaceutically acceptable carrier, excipients, diluent, surfactant, and/or vehicles.

The pharmaceutical composition may comprise a CD6-ALCAM pathway inhibitor and one or more agent selected from the group consisting of carriers, excipients, diluents, antioxidants, preservatives, coloring, flavoring and diluting agents, emulsifying agents, suspending agents, solvents, fillers, bulking agents, buffers, delivery vehicles, tonicity agents, cosolvents, wetting agents, complexing agents, buffering agents, antimicrobials, and/or surfactants. Such agents are known in the art (see, e.g., Remington's Pharmaceutical Sciences, 18th edition, Mack Publishing Co., Easton, Pa. (1990), incorporated herein by reference in its entirety.

The pharmaceutical composition may comprise EQ001 and one or more agent selected from the group consisting of carriers, excipients, diluents, antioxidants, preservatives, coloring, flavoring and diluting agents, emulsifying agents, suspending agents, solvents, fillers, bulking agents, buffers, delivery vehicles, tonicity agents, cosolvents, wetting agents, complexing agents, buffering agents, antimicrobials, and/or surfactants. Such agents are known in the art (see, e.g., Remington's Pharmaceutical Sciences, 18th edition, Mack Publishing Co., Easton, Pa. (1990), incorporated herein by reference in its entirety.

The present invention also includes combination therapies comprising administering to a patient a CD6-ALCAM pathway inhibitor such as an anti-CD6 antibody (e.g., or EQ001), or an antigen binding portion thereof in combination with a second active agent, or a device or a procedure capable of treating, preventing, or attenuating one or more asthma related symptom. In this context “administered in combination” means: (1) part of the same unitary dosage form; (2) administration separately, but as part of the same therapeutic treatment program or regimen, typically but not necessarily, on the same day.

In some aspects of these combination therapies, the second active agent is one or more agent capable of modulating the immune system. In some aspects of these combination therapies, the second active agent is one or more immunosuppressant.

In certain aspects, the CD6-ALCAM pathway inhibitor is an anti-ALCAM antibody or an antigen-binding portion. In some such instances, the anti-ALCAM antibody that blocks the binding of ALCAM to CD6. In some embodiments, the inhibitor is a small molecule inhibitor of the CD6-ALCAM pathway, e.g., a competitive or allosteric inhibitor.

As previously noted, the CD6-ALCAM pathway inhibitor may be administered alone as a monotherapy in some aspects or as a combination therapy in some aspects. In some aspects, any one of the CD6-ALCAM pathway inhibitors described herein (e.g., EQ001) for administering to a patient according to the methods disclosed herein may be administered in combination with one or more other therapeutic agent as a combination therapy. For example, a CD6-ALCAM pathway inhibitor (e.g., EQ001 or an anti-ALCAM antibody) may be administered to a patient as a combination therapy with another agent for the treatment of an inflammatory or autoimmune disease. The combination therapy may comprise administration of a CD6-ALCAM pathway inhibitor (e.g., EQ001 or an anti-ALCAM antibody) and any other anti-inflammatory or autoimmune disease therapeutic agent known in the art or disclosed herein. For example, the CD6-ALCAM pathway inhibitor (e.g., EQ001 or an anti-ALCAM antibody) may be administered to the subject in combination with an agent selected from, e.g., but not limited to, a steroid or an immunosuppressant. The steroid may be a corticosteroid. The corticosteroid may be prednisone. The CD6-ALCAM pathway inhibitor (e.g., EQ001 or an anti-ALCAM antibody) may be administered to the subject in combination with an agent selected from, e.g., but not limited to mycophenolate and cyclophosphamide.

In particular embodiments, EQ001 is administered to the subject in combination with an agent selected from, e.g., but not limited to, a steroid or an immunosuppressant, a corticosteroid, prednisone, mycophenolate and cyclophosphamide. EQ001 may also be administered to the subject in combination with an anti-ALCAM antibody.

The CD6-ALCAM pathway inhibitor (including, e.g., EQ001) may be administered before, after, or concurrently with one or more of such anti-inflammatory or autoimmune disease agents. In some embodiments, such combinations may offer significant advantages, including additive or synergistic activity in therapy.

In various embodiments, the compositions and methods disclosed herein, e.g., the methods for treating such inflammatory or autoimmune diseases discussed herein (e.g., SLE and LN), involve administering to a subject an effective amount of a CD6-ALCAM pathway inhibitor such as EQ001 or a composition (e.g., a pharmaceutical composition) comprising a CD6-ALCAM pathway inhibitor such as EQ001. The terms “CD6-ALCAM pathway inhibitor” and an “inhibitor of the CD6-ALCAM pathway” are used interchangeably herein to refer to any compound or substance that is capable of inhibiting signaling through the CD6-ALCAM pathway. These terms include, without limitation anti-CD6 antibodies described herein, as well as other inhibitors that are able to decrease or prevent signaling through CD6 for example anti-ALCAM antibodies. Non-limiting examples of anti-CD6 antibodies are known in the art and are disclosed herein. For example, but not to be limited in any way, in some embodiments, the compositions and methods described herein may utilize EQ001 as the CD6-ALCAM pathway inhibitor.

The CD6-ALCAM pathway inhibitor may be administered as a pharmaceutical composition. The CD6-ALCAM pathway inhibitor may be administered before, after, and/or concurrently with the one or more other therapeutic. If administered concurrently with the one or more other therapeutic agent, such administration may be simultaneous (e.g., in a single composition) or may be via two or more separate compositions, optionally via the same or different modes of administration (e.g., local, systemic, oral, intravenous, etc.).

Administration of the disclosed CD6-ALCAM pathway inhibitors and/or other therapeutic agents can be accomplished via any mode of administration for therapeutic agents. These modes include systemic or local administration such as oral, nasal, parenteral, transdermal, subcutaneous, vaginal, buccal, rectal or topical administration modes.

For administration in the methods of use described herein, the CD6-ALCAM pathway inhibitors, such as EQ001, may be mixed, prior to administration, with a non-toxic, pharmaceutically acceptable carrier substance (e.g. normal saline or phosphate-buffered saline), and will be administered using any medically appropriate procedure, e.g., parenteral administration (e.g., injection) such as by intravenous or intra-arterial injection.

Formulations of the CD6-ALCAM pathway inhibitors, such as EQ001, used in accordance with the present invention may be prepared by mixing an antibody having the desired degree of purity with optional pharmaceutically acceptable carriers, excipients or stabilizers in either the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives such as octadecyl dimethyl benzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol and m-cresol; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™, or polyethylene glycol (PEG).

The CD6-ALCAM pathway inhibitors, such as EQ001, may also be entrapped in microcapsules prepared, for example, by coacervation techniques or by interfacial polymerization, for example, hydroxy methyl cellulose or gelatin-microcapsules and poly-(methyl methacrylate) microcapsules, respectively, in colloidal drug delivery systems (for example, liposomes, albumin microspheres, microemulsions, nano-particles and nanocapsules) or in macroemulsions. Such techniques are well known in the art.

Sustained-release preparations may be prepared. Suitable examples of sustained-release preparations include semipermeable matrices of solid hydrophobic polymers containing the CD6-ALCAM pathway inhibitors, such as EQ001, which matrices are in the form of shaped articles, e.g. films, or microcapsules. Examples of sustained-release matrices include polyesters, hydrogels, copolymers of L-glutamic acid, non-degradable ethylene-vinyl acetate and degradable lactic acid-glycolic acid copolymers.

The CD6-ALCAM pathway inhibitors, such as EQ001, may be administered to a subject in accord with known methods, such as intravenous administration as a bolus or by continuous infusion over a period of time, by intramuscular, intraperitoneal, intracerobrospinal, subcutaneous, intra-articular, intrasynovial, intrathecal or oral routes. Intravenous or subcutaneous administration of the CD6-ALCAM pathway inhibitors, such as EQ001, is preferred.

Depending on the intended mode of administration, the disclosed compounds or pharmaceutical compositions can be in solid, semi-solid or liquid dosage form, such as, for example, injectables, tablets, suppositories, pills, time-release capsules, elixirs, tinctures, emulsions, syrups, powders, liquids, suspensions, or the like, sometimes in unit dosages and consistent with conventional pharmaceutical practices. Likewise, they can also be administered in intravenous (both bolus and infusion), intraperitoneal, subcutaneous or intramuscular form, and all using forms well known to those skilled in the pharmaceutical arts. Pharmaceutical compositions suitable for the delivery of a CD6-ALCAM pathway inhibitor (alone or, e.g., in combination with another therapeutic agent according to the present disclosure) and methods for their preparation will be readily apparent to those skilled in the art. Such compositions and methods for their preparation may be found, e.g., in Remington's Pharmaceutical Sciences, 19th Edition (Mack Publishing Company, 1995), incorporated herein in its entirety.

The dosage regimen utilizing the CD6-ALCAM pathway inhibitor is selected in accordance with a variety of factors including type, species, age, weight, sex and medical condition of the patient; the severity of the condition to be treated; the route of administration; the renal or hepatic function of the patient; and the particular disclosed compound employed. A physician or veterinarian of ordinary skill in the art can readily determine and prescribe the effective amount of the drug required to prevent, counter or arrest the progress of the condition.

An exemplary, non-limiting range for a therapeutically effective amount of the CD6-ALCAM pathway inhibitor (e.g., EQ001) used in the present invention is about 0.01-100 mg/kg per subject body weight, such as about 0.01-50 mg/kg, for example about 0.01-25 mg/kg. A medical professional having ordinary skill in the art may readily determine and prescribe the effective amount of the pharmaceutical composition required. For example, a physician could start doses of the CD6-ALCAM pathway inhibitor (e.g., EQ001) at levels lower than that required in order to achieve the desired therapeutic effect and gradually increase the dosage until the desi red effect is achieved.

In one embodiment the CD6-ALCAM pathway inhibitor (e.g., EQ001) is administered by infusion in a weekly dosage of from 1 to 500 mg kg per subject body weight such as, from 20 to 200 mg/kg. Such administration may be repeated, e.g., 1 to 8 times, such as 3 to 5 times. In the alternative, the administration may be performed by continuous infusion over a period of from 2 to 24 hours, such as, from 2 to 12 hours.

In one embodiment the CD6-ALCAM pathway inhibitor (e.g., EQ001) is administered in a weekly dosage of from 0 mg to 200 mg, for up to 7 times, such as from 4 to 6 times. The administration may be performed by continuous infusion over a period of from 2 to 24 hours, such as, from 2 to 12 hours. Such regimen may be repeated one or more times as necessary, for example, after 6 months or 2 months.

In some embodiments, the present disclosure also provides kits for performing the methods described herein. Suitable kits may comprise reagents sufficient for performing an assay for detecting and/or quantifying amount of one or both of soluble CD6 and soluble ALCAM in a sample, together with instructions for performing the detection assay and optional threshold comparisons. Such kits may also comprise reagents sufficient for performing an assay for detecting additional markers, for example other proteins. For example, the kits may comprise reagents for detecting and/or quantifying soluble CD6 and/or soluble ALCAM by performing a single-plex ELISA; multiplex ELISA, bead-based immunocapture with FACs-based detection; bead-based immunocapture with ELISA-based detection; bead-based immunocapture with chemiluminescent-based detection; meso-scale diagnostic (MSD); quantitative western blot; high performance liquid chromatography (HPLC); or a combination thereof. Such may include, without limitation, antibodies, or antigen-binding portions thereof, directed to soluble CD6 and/or soluble ALCAM, or portions of soluble CD6 and/or soluble ALCAM. Such kits may also include, without limitation, aptamers that bind to soluble CD6 and/or soluble ALCAM, or portions of soluble CD6 and/or soluble ALCAM. The kits may also comprise reagents for performing sample preparation, e.g., buffers, reagents, tubes, and the like for obtaining samples, purifying samples, storing samples (e.g., refrigerating or freezing samples), etc. The kits may also comprise control samples (such as standard control CD6 and/or ALCAM protein samples of known concentration) for use in generating standard curves to quantify the levels of soluble CD6 and/or soluble ALCAM that are detected in a subject's sample. The kits may provide one or more antibody pair for performing a sandwich assay, or a labeled species for performing a competitive assay, for the analyte. The antibody pair may comprise a first antibody conjugated to a solid phase and a second antibody conjugated to a detectable label, wherein each of the first and second antibodies bind soluble CD6 or soluble ALCAM. The antibodies in the antibody pairs may be monoclonal.

All of the U.S. patents, U.S. patent application publications, U.S. patent applications, PCT patent application, PCT patent application publications, foreign patents, foreign patent applications and non-patent publications referred to in this specification or listed in any Application Data Sheet are incorporated herein by reference in their entirety. From the foregoing it will be appreciated that, although specific embodiments of the invention have been described herein for purposes of illustration, various modifications may be made without deviating from the spirit and scope of the invention.

### EXAMPLES

The disclosure is further illustrated by the following examples and synthesis examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### Example 1

### ELISA-Based Detection of Soluble CD6 and Soluble ALCAM Protein in Urine of Subjects with Lupus Nephritis

Unless otherwise stated, the Examples disclosed herein utilize the following materials and methods.

30 ml of urine is collected from a patient (or control individual) and treated with a protease inhibitor cocktail tablet on ice (MiliporeSigma, Burlington, Mass., USA) according to manufacturer's instructions. The sample is centrifuged at 10,000×g for 1 min or 5,000×g for 2 min, and the urine is removed from any pellet, aliquoted, and quick frozen in a dry ice/methanol bath and stored at −80° C. until use.

Several suitable ELISA kits are available commercially and are suitable for use in detecting soluble ALCAM and soluble CD6. For example, human ALCAM DuoSet ELISA (R&D Systems, Minneapolis, Minn.) is utilized according to the manufacturer's instructions to measure soluble ALCAM in a urine sample, and quantification is performed by generating a standard curve. Human CD6 ELISA kit (Sandwich ELISA)(LifeSpan BioSciences, Inc, Seattle, Wash.) is utilized according to the manufacturer's instructions to measure soluble CD6 in a urine sample, and quantification is performed by generating a standard curve.

Samples are collected from the following cohorts:
(a) Cohort 1: 12 normal subjects that do not have any known or expected inflammatory or autoimmune diseases
(b) Cohort 2: 6 subjects that have known lupus nephritis that is inactive
(c) Cohort 3: 6 subjects that have active lupus nephritis

Urine samples are collected from the subjects biweekly over a time course of one year. Immediately after collection, samples are treated, aliquoted, and frozen in accordance with the above method. Several disease activity and damage indexes are monitored to assess clinical presentation of lupus nephritis over the course of the study (See, e.g., Balow J E, Lupus. 2005; 14(1):25-30, incorporated herein by reference in its entirety).

Initially, concentration of soluble CD6 and soluble ALCAM is compared between cohorts and found to be highest in Cohort 3 and lowest in Cohort 1. Intermediate concentrations are present in samples from Cohort 2.

Over the course of the study, increases in soluble CD6 and soluble ALCAM are found in samples from certain subjects in Cohort 2, and the increases correlate with these subject's transition from inactive to active disease. Conversely soluble CD6 and soluble ALCAM levels decrease in certain subjects in Cohort 3, as they transition from active disease to inactive disease.

Such results are found to support the use of soluble CD6 and soluble ALCAM as biomarker of active lupus nephritis.

Based on these results, a subsequent study is performed utilizing an expanded group of cohorts to investigate the use of urinary levels of soluble CD6 and soluble ALCAM as a biomarker of disease progression in other inflammatory kidney disease.

Samples are collected from the following cohorts:
(a) Cohort 1: 12 normal subjects that do not have any known or expected inflammatory or autoimmune diseases
(b) Cohort 2: 6 subjects/group that have known inflammatory kidney disease that is inactive
   (i) Cohort 2a: subjects with known inactive IgA nephropathy
   (ii) Cohort 2b: subjects with known inactive anti-neutrophil cytoplasmic antibody-associated glomerulonephritis
   (iii) Cohort 2c: subjects with known inactive autoimmune (formerly idiopathic) membranous nephropathy
   (iv) Cohort 2d: subjects with known inactive anti-glomerular basement membrane glomerulonephritis
   (v) Cohort 2e: subjects with known inactive C3 nephropathy
   (vi) Cohort 2f: subjects with known inactive lupus nephritis
(c) Cohort 3: 6 subjects/group that have active inflammatory kidney disease.
   (i) Cohort 2a: subjects with known active IgA nephropathy
   (ii) Cohort 2b: subjects with known active anti-neutrophil cytoplasmic antibody-associated glomerulonephritis
   (iii) Cohort 2c: subjects with known active autoimmune (formerly idiopathic) membranous nephropathy
   (iv) Cohort 2d: subjects with known active anti-glomerular basement membrane glomerulonephritis
   (v) Cohort 2e: subjects with known active C3 nephropathy
   (vi) Cohort 2f: subjects with known active lupus nephritis

Urine samples are collected from the subjects biweekly over a time course of one year and disease status is followed as described above. Immediately after collection, samples are treated, aliquoted, and frozen in accordance with the above method.

Comparisons across cohorts are performed as in the first study and the results for lupus nephritis are repeated and similar results are observed in the context of the other inflammatory kidney cohorts.

Such results are found to support the use of soluble CD6 and soluble ALCAM as biomarker of inflammatory kidney disease, and in particular, of active lupus nephritis, IgA nephropathy, anti-neutrophil cytoplasmic antibody-associated glomerulonephritis, autoimmune (formerly idiopathic) membranous nephropathy, anti-glomerular basement membrane glomerulonephritis, and C3 nephropathy.

### Example 2

### CD6 and ALCAM Expression are Significantly Elevated in Human Renal Tissue Biopsies and are Detectable in Urine Cells from Lupus Nephritis (LN) Patients

Background/Purpose: Lupus nephritis (LN) is a leading cause of morbidity and mortality in systemic lupus erythematosus (SLE) patients. However, the pathogenesis of renal disease in lupus patients is not yet fully understood. The objectives of this research were to study the expression of CD6/ALCAM in kidneys of LN patients and to evaluate the potential of urine ALCAM and CD6 as biomarkers in LN disease.

Summary of Results: De novo analysis of a publicly available RNASeq dataset confirms that lupus nephritis patients express significantly higher renal levels of CD6 and ALCAM as compared to non-diseased individuals, and the high expression of these markers is detectable in urine cells collected from the LN patients.

Methods and Results: Single cell RNA Seq data was obtained from [frozen renal tissue samples or cells obtained from urine samples] isolated from lupus nephritis patients or healthy control patients (biopsy) as a part of the AMP Lupus Network Project (SDY997), and datasets consisting of the counts of transcriptional reads that map to all individual genes for each sample was made available in a public database (Arazi A. et al., Nat Immunol. 2019 July; 20(7):(902-914)). scRNA-seq analysis was performed on these datasets using the Seurat package for R.

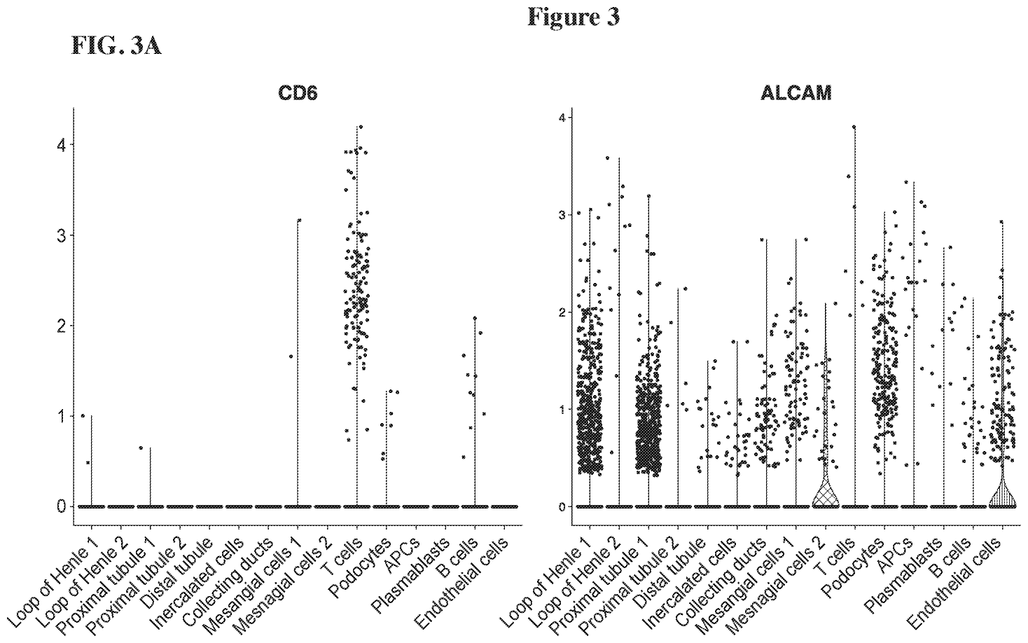

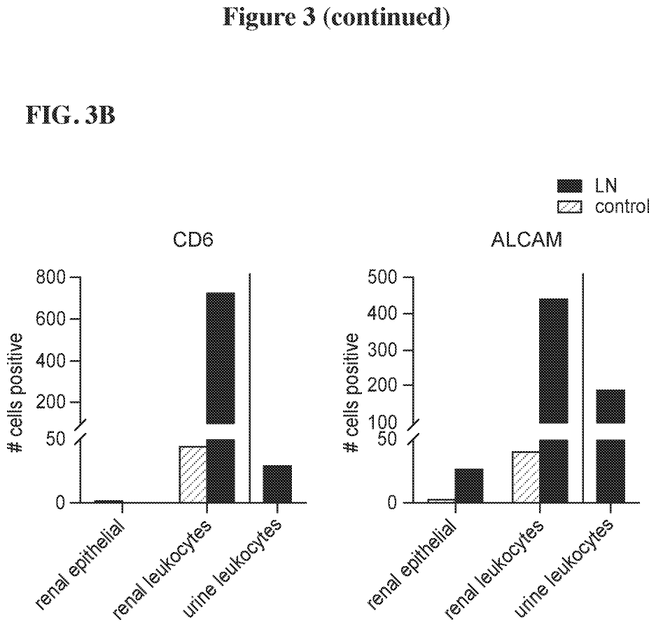

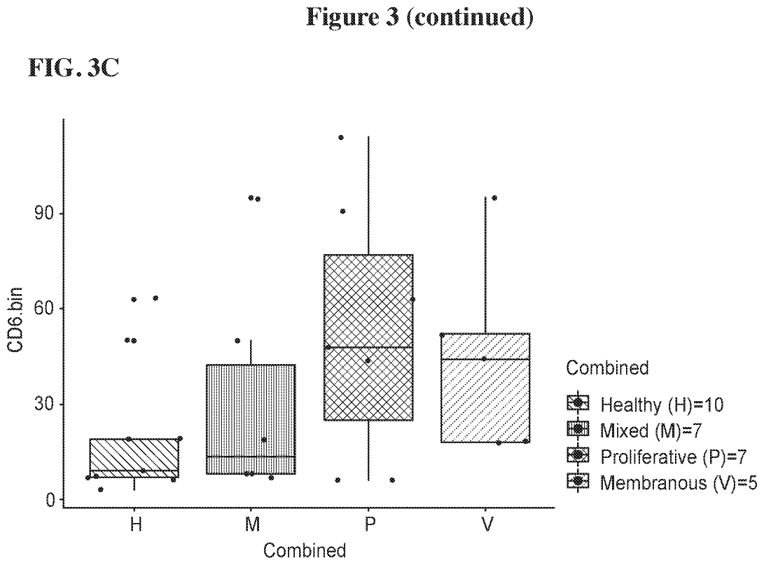

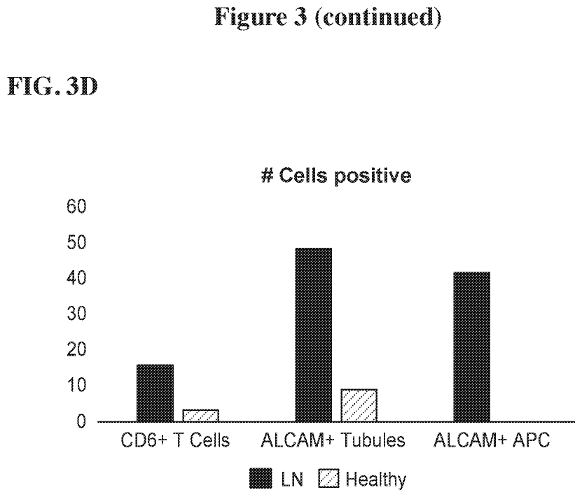

We mined these public datasets using bioinformatics to create comparisons of CD6 and ALCAM expression in control samples (11 healthy patients) vs. LN patient samples (19 LN patients). CD6 was exclusively expressed in T cells (FIG. 3A, left panel); whereas ALCAM was expressed in both professional APCs such as macrophages, dendritic cells, and tubular cells (FIG. 3A, right panel). Data from this experiment is summarized in Table 2, below.

TABLE 2
Expression profiles of CD6 and ALCAM across renal cell populations.

| Renal cell population | CD6 | ALCAM |
|---|---|---|
| Immune cells | | |
| T cells | ++ | +/− |
| APCs | − | + |
| B cells | +/− | +/− |
| Plasmablasts | − | +/− |
| Structural cells | | |
| Loop of Henle | − | ++ |
| Proximal tubule | − | ++ |
| Distal tubule | − | +/− |
| Intercalated cells | − | +/− |
| Collecting ducts | − | + |
| Mesangial cells | − | +/− |
| Endothelial cells | − | + |
| Podocytes | − | ++ |

Our analysis demonstrated the presence of the CD6 and ALCAM at greater levels in LN patients vs. controls (FIG. 3B). Both CD6+ and ALCAM+ leukocytes were found in greater numbers in the kidneys of LN patients vs. controls (FIG. 3B, “renal leukocytes” bar graphs on left and right panels) and ALCAM+ epithelial cells were only detectable in LN patients (FIG. 3B, “renal epithelial” bar graphs on left and right panels). Moreover, both CD6+ and ALCAM+ leukocytes were present in urine of LN patients (contrast to disease free patients, which are known to not have leukocytes present in their urine. (FIG. 3B, “urine leukocytes” bar graphs on left and right panels).

Patients with class III (Proliferative) or IV (Membanous) LN trended towards having more CD6 expressing cells than controls (FIG. 3C), suggesting that CD6 expression might follow LN stage. Furthermore, the number of ALCAM expressing tubular cells and macrophages were elevated in LN compared to healthy controls as well (FIG. 3D). Thus, these data suggest that LN patients have increased CD6+ T cells in the kidney due to increased T cell infiltration, and these T cells are likely activated given the increased ALCAM expression on both epithelial cells and on infiltrating renal leukocytes.

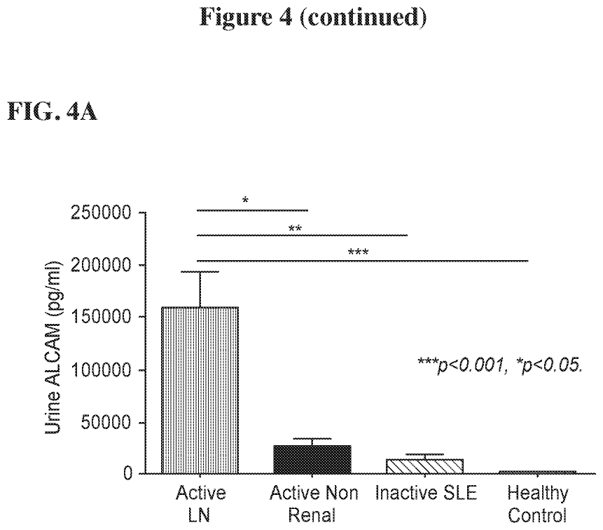

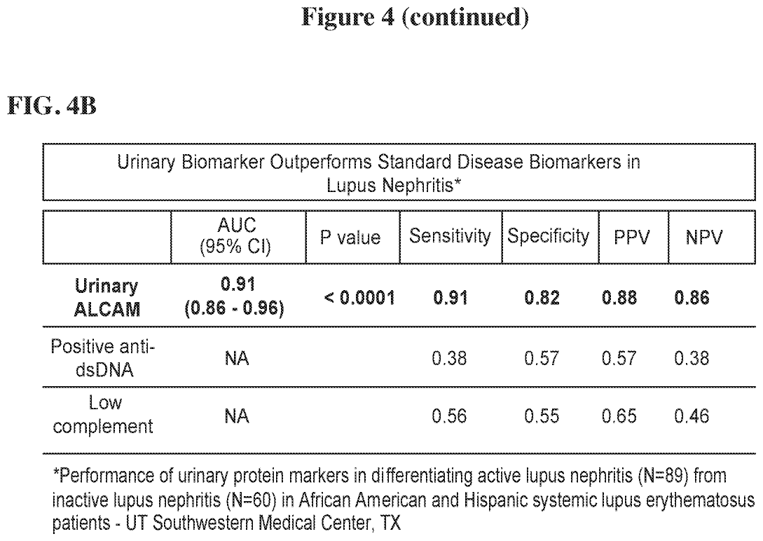

Urinary ALCAM protein levels were significantly elevated in active LN patients as compared to control individuals (FIG. 4A), and unbiased screening of >1100 urinary proteins identified urinary ALCAM as a strong predictor of LN disease activity in LN patients (FIG. 4B).

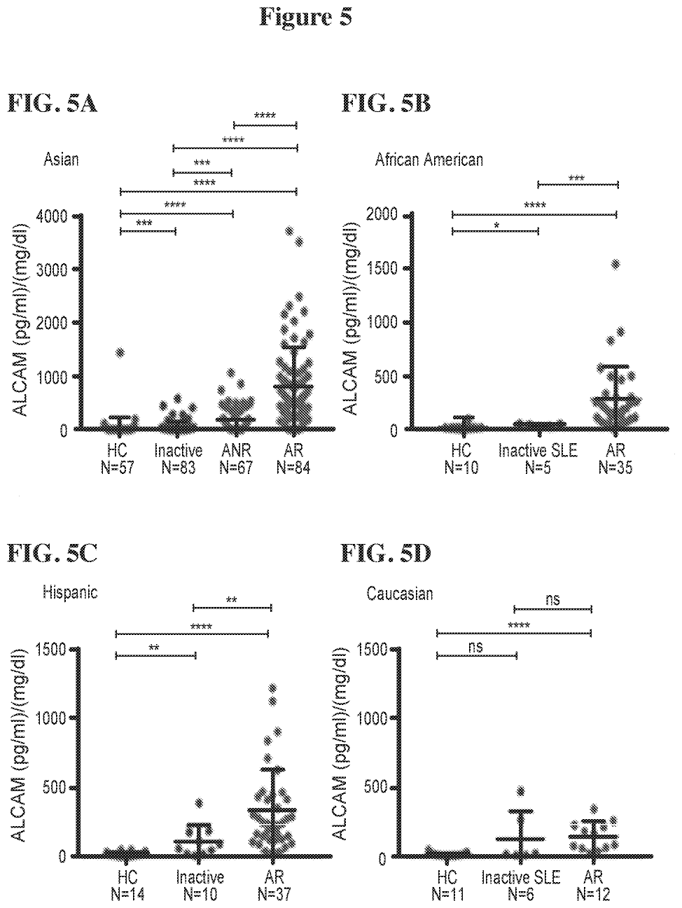

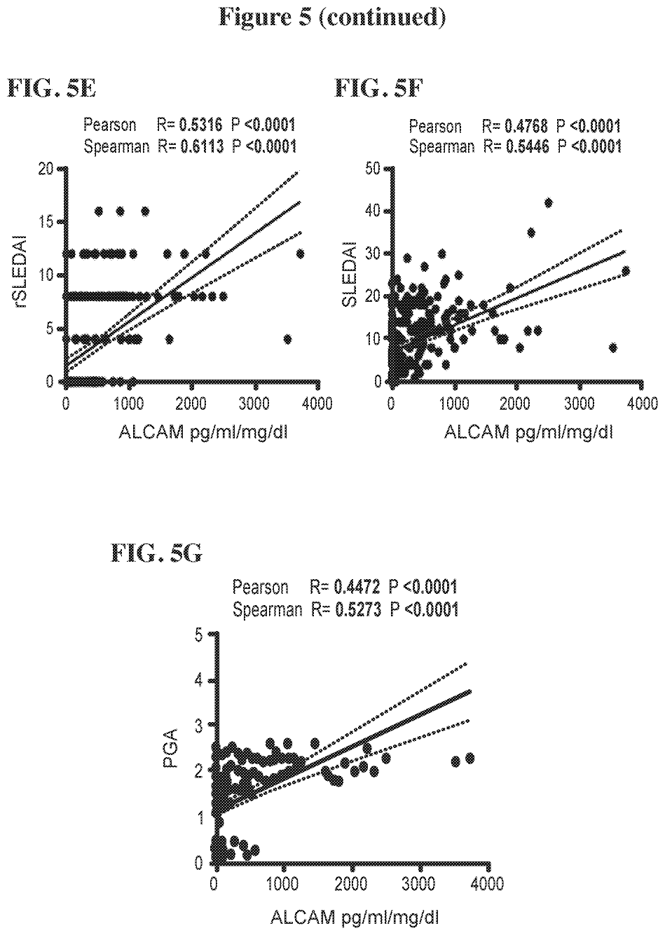

Urine samples were collected from SLE patients of multiple ethnicities and diverse disease activities. ALCAM concentrations were assayed by ELISA then normalized to urine creatinine. ALCAM was significantly elevated in urine from active LN patients when compared with controls of multiple ethnicities (FIG. 5). In Asian (FIG. 5A) African American (FIG. 5B), Hispanic (FIG. 5C), and Caucasian patients (FIG. 5D), urine ALCAM further discriminated active LN from inactive SLE or active SLE patients without LN. For FIGS. 5A-5D, HC=healthy controls; ANR=active non-renal lupus; AR=active renal lupus.

Urine ALCAM correlated significantly with Systemic Lupus Erythematosus Disease Activity Index (SLEDAI)(FIG. 5F), renal domains of SLEDAI (rSLEDAI)(FIG. 5E), and PGA (FIG. 5F) in Asian SLE patients (all p<0.0001) (FIG. 5).

Conclusion: Here, we demonstrate increased activity of the CD6/ALCAM pathway within the renal tissues of LN patients. More specifically, infiltrating T cells do indeed express CD6 and the number of CD6-expressing T cells are greater in renal biopsies from patients with LN vs. healthy controls, and in patients with proliferative vs. membranous LN. ALCAM-expressing macrophages were also numerically elevated in patients with LN, suggesting increased activation of the CD6/ALCAM signaling pathway in LN. Patients with LN also had elevated levels of ALCAM-expressing tubular cells, indicating these resident kidney cells may contribute to signaling and migration of T cells in the context of LN. Finally, urine ALCAM was significantly elevated in active LN patients in multiple ethnicities, and correlated well with clinical disease status, thus representing a promising biomarker for disease evaluation in LN, and CD6+ and ALCAM+ mRNA was detectable in leukocytes harvested from urine samples obtained from LN patients. These data strongly support that LN may be diagnosed and/or that the LN activity state and disease progression may be monitored by analyzing the presence and abundance of ALCAM or CD6 protein in urine and/or mRNA expression in urinary lymphocytes, and further suggest that a targeted CD6-ALCAM therapy, such as itolizumab, may be a promising treatment for LN.

### Example 3

### High Levels of Soluble CD6 and Soluble ALCAM Protein in Urine as a Biomarker for Sensitivity to Treatment with EQ001

In order to determine whether high urinary levels of soluble CD6 and soluble ALCAM can be used as a biomarker for sensitivity to treatment with EQ001, a clinical trial is performed in which concentrations of these markers are analyzed in the urine of subjects with active lupus nephritis before and after treatment with EQ001.

Samples are collected from the following cohorts:
(a) Cohort 1: 12 normal subjects that do not have any known or expected inflammatory or autoimmune diseases, treated with vehicle
(b) Cohort 2: 6 subjects that have known lupus nephritis that is inactive, treated with vehicle
(c) Cohort 3: 6 subjects that have active lupus nephritis, treated with vehicle
(d) Cohort 4: 6 subjects that have active lupus nephritis, treated with EQ001 delivered in vehicle.

Urine samples are collected from the subjects biweekly for 2-3 months to establish individual baseline urinary soluble CD6 and soluble ALCAM concentrations. Treatment is initiated after at least five baseline measurements are obtained. Subjects receive EQ001 or vehicle administered intravenously every two weeks for a total of 5 doses, and a urine sample is collected 3 days after initial dosing, and twice weekly thereafter over the course of the study, which continues for 20 weeks post-initial treatment.

As described above, immediately after collection, samples are treated, aliquoted, and frozen in accordance with the above method, and upon the completion of the study, samples are tested by ELISA.

Initially, concentration of soluble CD6 and soluble ALCAM is compared between cohorts and found to be highest in Cohorts 3 and 4 and lowest in Cohort 1. Intermediate concentrations are present in samples from Cohort 2.

Over the course of the study, increases in soluble CD6 and soluble ALCAM are found in some samples from certain subjects in Cohort 2, and the increases correlate with these subject's transition from inactive to active disease. Conversely significant decreases in soluble CD6 and soluble ALCAM levels are observed in subjects in Cohort 4, which received EQ001. In contrast, no significant decreases are observed in Cohort 3, which received vehicle alone. Subjects with the highest basal levels of soluble CD6 and soluble ALCAM respond most dramatically to treatment with EQ009.

Such results are found to support the use of soluble CD6 and soluble ALCAM as biomarker for sensitivity to treatment with an inhibitor of the CD6-ALCAM pathway such as the anti-CD6 antibody EQ001.

### Example 4

### Treatment of Spontaneous SLE/Lupis Nephritis (LN) with CD6 Blockade

Background/Purpose: In order to determine whether a subject might beneficially respond to treatment with an anti-CD6 blocking antibody, e.g., after diagnosis with LN according to the present disclosure, we performed various in vivo studies using the MRL/MpJ-Faslpr/2J mouse strain (or MRL/lpr), an extensively used model of SLE and LN. This strain develops spontaneous systemic autoimmunity with many similarities to human SLE and LN disease and is routinely used in SLE/LN research (Richard 2018). This strain contains a mutation in the fas gene that results in loss of apoptosis and uncontrolled lymphoproliferation characterized by systemic autoimmunity, lymphadenopathy, and hyperactive T and B cells. Analogous to SLE patients, mice develop autoantibodies against nuclear antigens (anti-nuclear antibodies, anti-dsDNA, anti-Sm, anti-Ro and anti-La), deposition of immune complexes, glomerulonephritis, and additional SLE manifestations including arthritis, cerebritis, and skin rash. In this study, we assessed the expression of CD6 and ALCAM within the context of this murine model of SLE, and then subsequently targeted this signaling axis to determine its role in the pathogenesis of disease.

We first analyzed renal CD6 and ALCAM expression in the MRL/MpJ-Faslpr/2J mouse strain to confirm the overexpression observed in human samples is recapitulated in this model. In an initial experiment, 6 month old MRL/lpr mice and B6 kidneys were stained for the presence of both ALCAM and CD6.

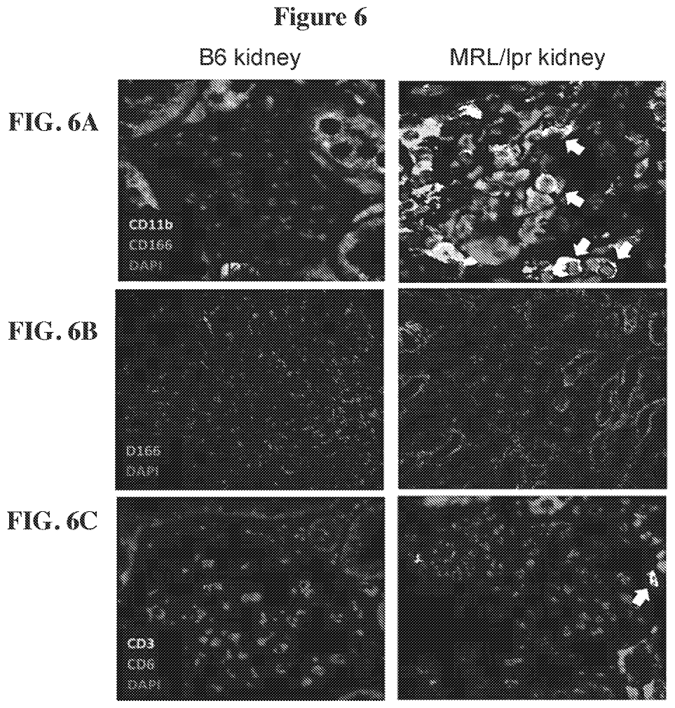

Kidneys were harvested from MRL/lpr mice (which have nephritis) and C57BL/6 mice (which do not have nephritis) at 6 months of age were stained for ALCAM (CD166, red, FIG. 6A and FIG. 6B) and CD6 (red, FIG. 6C). MRL/lpr mice show increased levels of renal ALCAM expression, both within their tubules (FIG. 6B) and glomeruli (FIG. 6A) compared to B6 healthy control mice (shown are images representative of 3 mice per group). Additionally, macrophages infiltrating into the glomeruli of MRL/lpr mice were ALCAM+(white arrows, top panel) and were paired with a concomitant increase in CD6+ T cell infiltration (white arrows, bottom panel).

Thus, immunofluorescence staining indeed confirmed that CD6 and ALCAM were present at greater levels in the kidneys of animals with nephritis than the levels seen in non-nephritic kidneys. Accordingly, these mice recapitulated the expression patterns observed in human LN tissues presented in the above Examples, and these mice were used to study the effects of treatment with a monoclonal anti-CD6 antibody capable of blocking signaling through the CD6/ALCAM pathway.

Several independent experiments were performed to test reproducibility and a wide range of endpoints. The design of these experiments was in keeping with accepted practices of preclinical testing of therapeutics in LN.

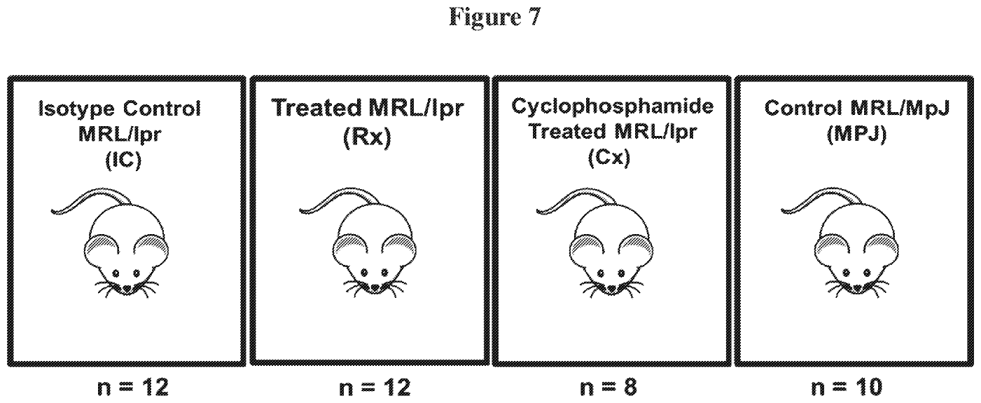

FIG. 7A shows the study design of the experiments using the MRL/lpr model. Briefly, female MRL/lpr mice were aged to 9-10 weeks of age, after which mice were treated with either anti-CD6 antibody (10D12, 60 ug/dose, intraperitoneally twice per week), an irrelevant polyclonal rat IgG isotype control (60 ug/dose, twice per week), or cyclophosphamide (25 mg/kg, once per week). We also included a no treatment group and a group of MRL/MpJ mice, a congenic healthy control strain. Proteinuria, weights, and lymphadenopathy were monitored in-life, while terminal endpoints included urine albumin and creatinine levels, lymph node and spleen weights, and renal-infiltrating immune cells.

Baseline levels of anti-DNA antibodies, weight, and proteinuria in the MRL/lpr groups were similar (data not shown). Mice were monitored weekly for proteinuria, lymph node swelling, and macroscopic skin lesions.

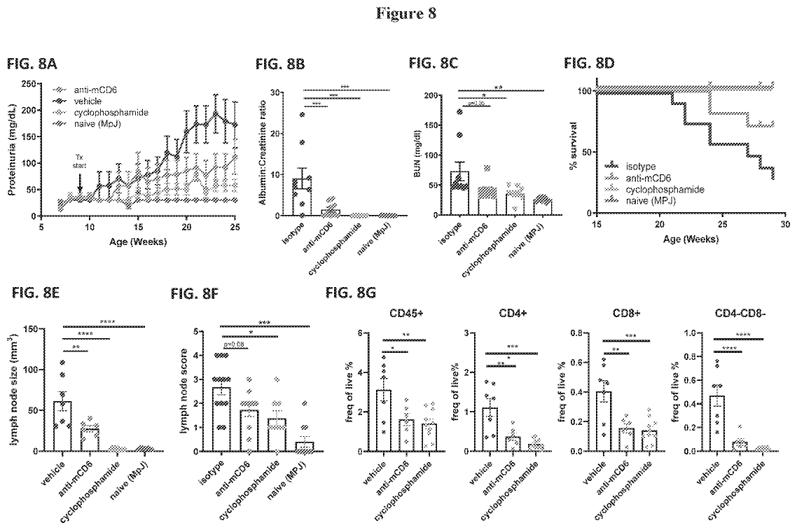

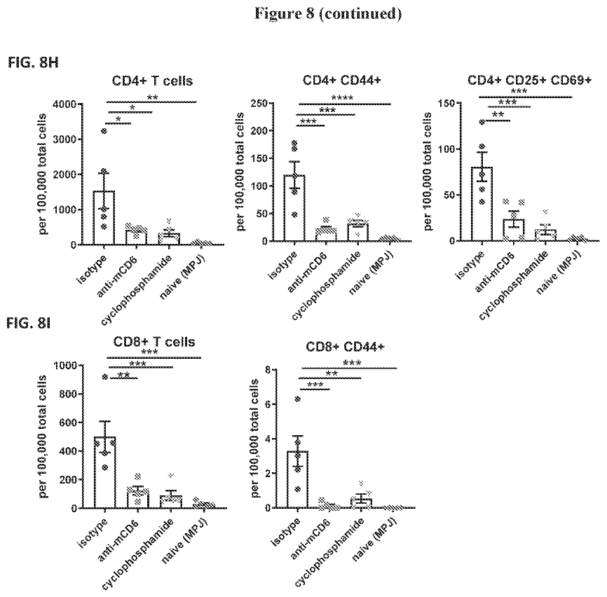

As shown in FIG. 8 administration of anti-CD6 antibody decreased kidney damage resulting in improved renal function and decreased mortality. Specifically, At 19 weeks of age, mice treated with anti-CD6 antibody showed improved proteinuria compared to isotype control mice as measured by uristix (FIG. 8A, p<0.05) and as confirmed by measuring albumin:creatinine ratios in terminal urine (FIG. 8B). Treatment with anti-CD6 antibody also improved kidney function of MRL/lpr mice as measured by blood urea nitrogen (BUN) levels in terminal serum (FIG. 8C), and the mice treated with anti-CD6 antibody showed significant improvements in survival (FIG. 8D).

MRL/lpr mice develop lymphoproliferative disease which results in abnormally large lymph nodes. Assessing lymphadenopathy at 19 weeks of age, we noted a marked improvements in the anti-CD6 treated mice, compared to controls, as assessed by average of the volume measurement of the left and right inguinal lymph nodes at termination (FIG. 8E) and scoring of lymph node swelling (FIG. 8F). Moreover, the frequency of kidney infiltrating immune cells and T cells at termination was reduced by anti-mCD6 treatment (FIG. 8G, FIG. 8H, and FIG. 8I); thus, demonstrating that CD6 blockade decreases the number of activated renal-infiltrating T cells.

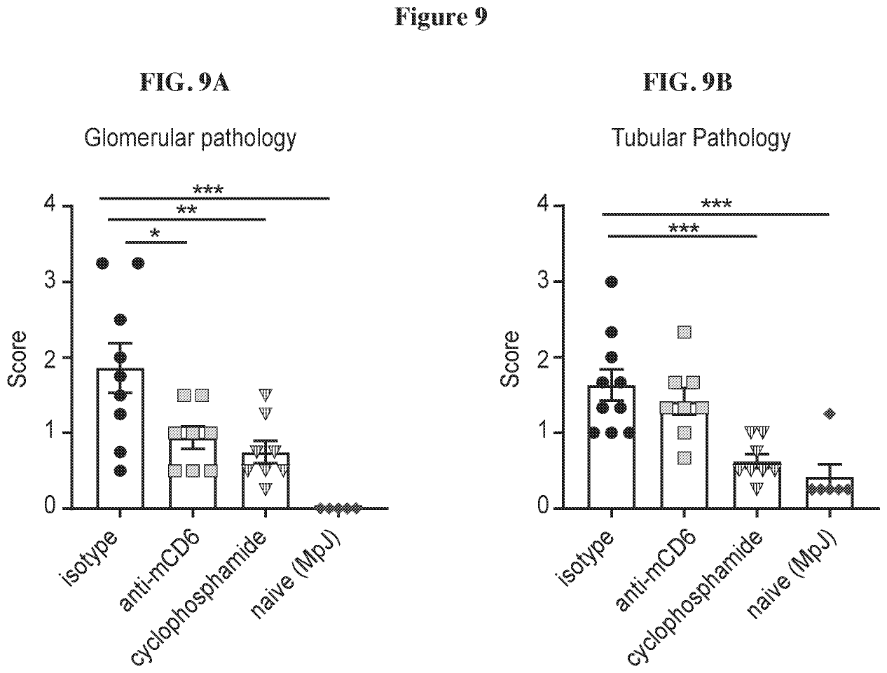

Furthermore, histological scoring of a glomeruli (FIG. 9A) and renal tubules (FIG. 9B) in samples from anti-CD6 antibody-treated mice and control mice was conducted by a blinded pathologist, and data demonstrated significant improvements in glomerular pathology.

Thus, these data demonstrate that the administration of anti-CD6 antibody decreased kidney damage resulting in improved renal function and mortality.

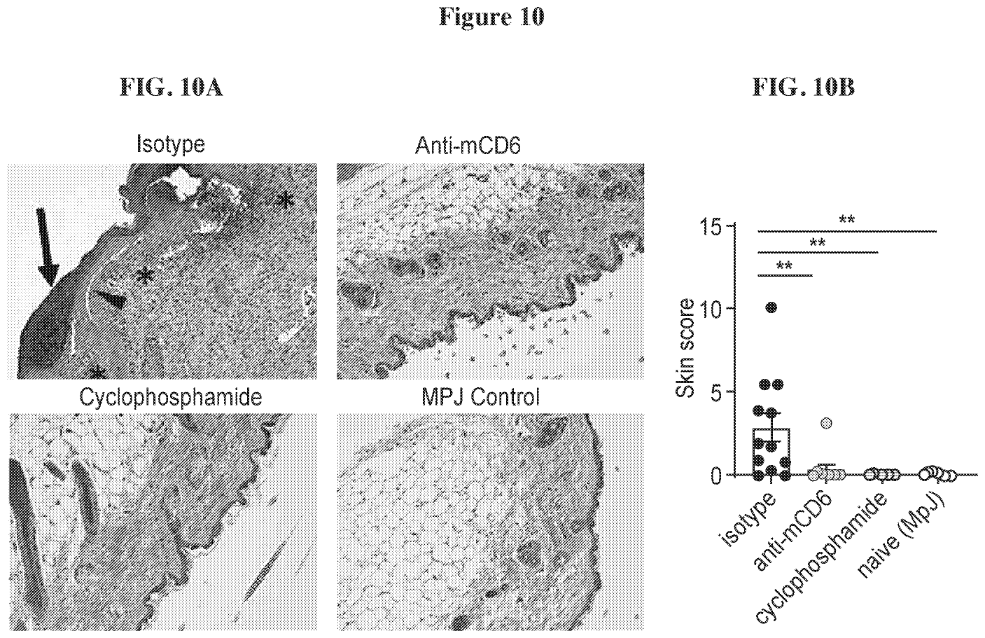

MRL/lpr mice also develop severe skin lesions, which are similar in pathology to cutaneous lupus seen in SLE patients, and which are the result of auto-inflammatory disease. FIG. 10A shows histological examination of skin tissue from control mice and from mice that received anti-CD6 antibody treatment. Isotype control mice displayed diseased skin histopathology, including hyperkeratosis (thickening of the epidermis), damage to the dermal-epidermal junction, and large cellular infiltrates into the dermis. Anti-CD6 treatment ameliorated many of these pathologies, demonstrating reduced epidermal thickening and cellular infiltrates. Anti-CD6 histology is more similar to healthy control sections from MPJ mice than to the isotype control mice. Macroscopic scoring of these lesions showed a significant improvement in the skin disease of the anti-CD6 treated mice compared to the isotype control group (FIG. 10B, p<0.05).

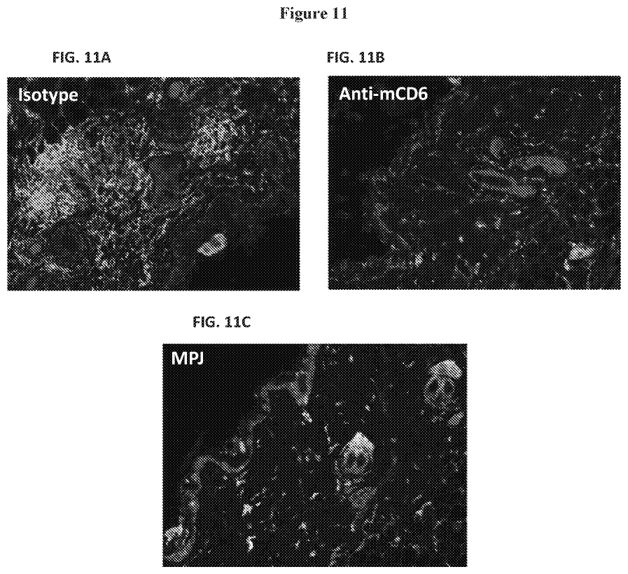

To assess how anti-CD6 treatment affected the development of skin disease in the MRL/lpr mice, skin tissue sections were stained for macrophages (green), C3 (red), and IgG (orange). There is a noticeable decrease in the number of accumulating macrophages in treated mice (FIG. 11B) compared to isotype control mice (FIG. 11A). C3 and IgG levels were similar between both treatment groups, however, and appear higher than the healthy control MPJ mice (FIG. 11C).

Conclusion: Within a spontaneous model of SLE, anti-CD6 treatment ameliorated multiple end organ pathologies, namely in the kidney and skin, while also significantly reducing the lymphoproliferative phenotype of this model. Overall, these results indicate that targeting the CD6-ALCAM pathway may have promising therapeutic potential for multiple end organ pathologies within SLE.

### Example 5

### Treatment of SLE/Lupis Nephritis (LN) with CD6 Blockade in Accelerated Mouse Model of Nephrotoxic Serum Nephritis (NTN)

Background/Purpose: To examine the role of CD6 blockade and specific effects on the kidney, we utilized an accelerated model of nephrotoxic serum nephritis (NTN). NTN is a validated, short-term model of LN. The NTN model exhibits glomerulonephritis that is mechanistically and histologically similar to that observed with LN and, consequently is commonly used as a model to test pharmacologic agents for this specific complication of SLE (Fu 2007). Animals exhibit crescentic, proliferative glomerulonephritis characterized by immune complex deposition, complement activation, and immune cell infiltration (T cells, neutrophils, and macrophages), accompanied by a decreased glomerular filtration rate, proteinuria, and albuminuria, features all similar to human disease.

The NTN model initiates rapid-onset immune complex disease in nonautoimmune mice via injection of an antibody to the glomerular basement membrane (anti-GBM). Complement-mediated damage is followed by infiltration of T cells into the kidney and T cell-mediated destruction.

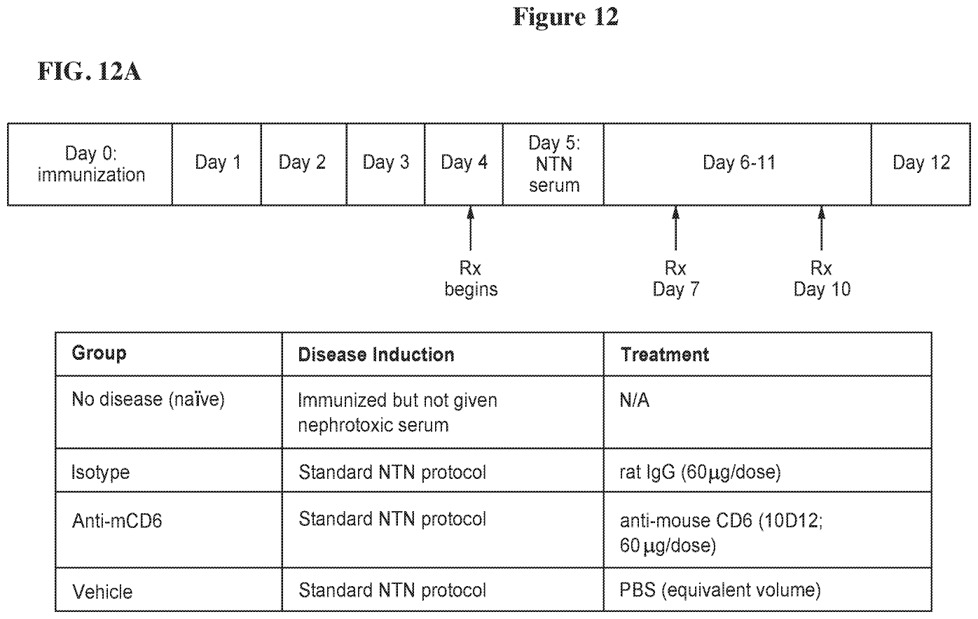

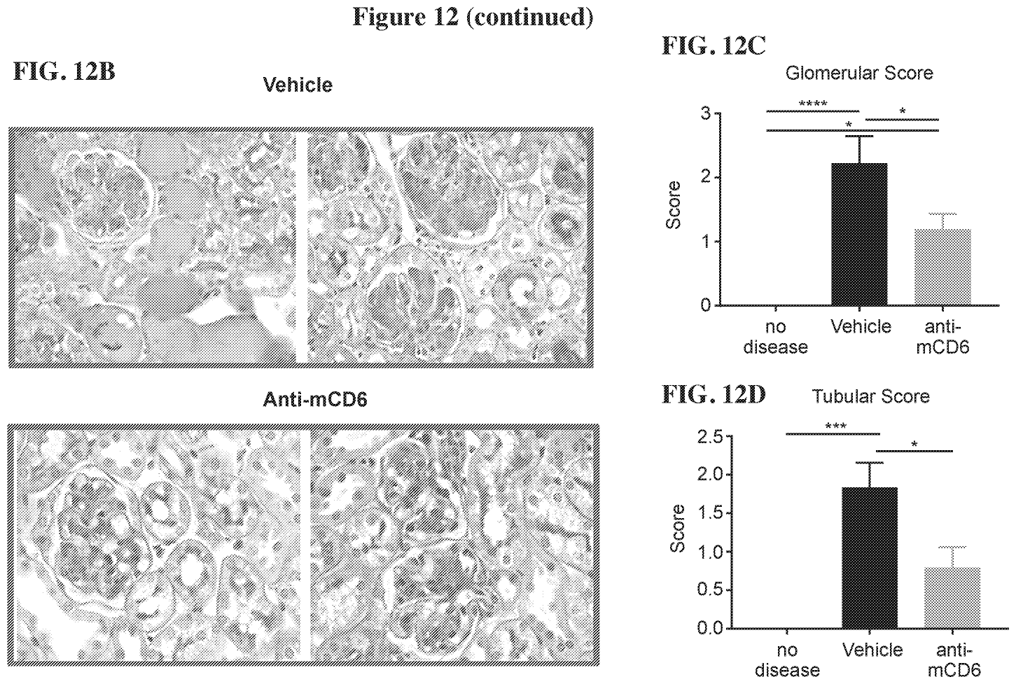

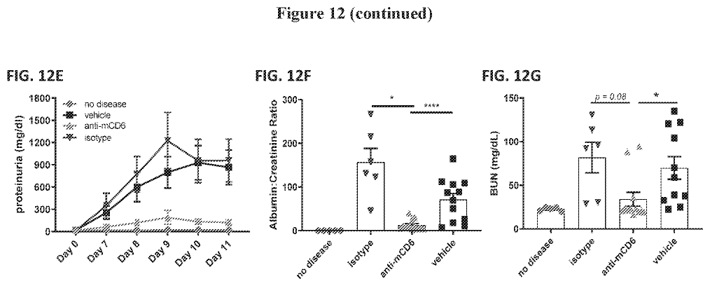

Two independent experiments were performed to test reproducibility and examine a wide range of endpoints in a manner standard to the preclinical evaluation of therapeutics for LN (n=6-12 per group/experiment). In Experiment 1, mice were treated with either vehicle control or a 60 μg/dose of anti-mCD6 (10D12). In Experiment 2, mice were treated with vehicle or a 60 μg/dose of either anti-mCD6 or isotype control. The treatment schedule for each of Experiments 1 and 2 is depicted in FIG. 12A. Treatment began one day before injection of the rabbit serum (Day 4) and was administered every 3 days until sacrifice (Experiment 1: Day 12; Experiment 2: Day 11); the sacrifice was set at the second day after peak proteinuria as determined by daily tracking. Both experiments included healthy (no disease-initiation) mice as controls.

Methods: Nephrotoxic serum nephritis was induced in two separate cohorts of female 129/svJ mice, both aged to 10 weeks, to model LN. Mice were immunized with rabbit IgG and CFA on day 0 to create mouse anti-rabbit antibodies, which then cross-reacted with nephrotoxic rabbit serum given on day 5, causing an antibody-mediated nephritis similar in pathology to LN. To assess the importance of the CD6/ALCAM pathway in LN pathogenesis, mice were treated on Days 4, 7, and 10 with an anti-CD6 monoclonal antibody (mAb) (60 ug/dose, n=12/experiment), or with vehicle or isotype IgG (n=12/experiment). Healthy mice (immunized with rabbit IgG, but not given nephrotoxic serum) were also included as a control (n=12). We monitored the progress of kidney disease via proteinuria (uristix), urinary albumin:creatinine ratio, and serum blood urea nitrogen (BUN) to assess the effect of the anti-CD6 treatment on both cohorts. To assess the effect of treatment on immune cell infiltration, flow cytometry, RT-PCR, and immunofluorescent staining was completed at termination.

Results: CD6 blockade in the NTN murine model of nephrotoxic serum nephritis inhibits disease and protects kidney function (FIG. 12B-FIG. 12G). FIG. 12B shows histological glomerular sections of renal tissue from mice treated with vehicle control (top panel) or the CD6 antibody (bottom panel). Glomerular sections were assessed via blinded scoring by an experienced nephropathologist of endocapillary proliferation, crescent, and deposits on a scale from 0-4, and the results are graphed in FIG. 12C. Anti-CD6 treatment significantly attenuated glomerular pathology vs. vehicle control mice. Tubular scores were similarly determined by scoring tubular casts and interstitial inflammation on a scale 0-4, and the results are graphed in FIG. 12D. Like the glomerular scores, anti-mCD6 treated mice exhibited significantly improved tubular score compared to vehicle control.

Further, treatment of the NTN mice with the anti-CD6 mAb resulted in decreased levels of proteinuria (p<0.001) compared to vehicle control mice (FIG. 12E). This result was confirmed by measuring albumin:creatinine ratios in terminal urine (FIG. 12F, p<0.0001). We also found a significantly improved BUN (p<0.01) when comparing treated mice to vehicle control mice (FIG. 12G). To ensure that anti-CD6 treatment did not interfere with the induction of the NTN model, we measured mouse anti-rabbit IgG levels and rabbit anti-mouse glomerular basement membrane (GBM) levels and found no difference between the groups (data not shown).

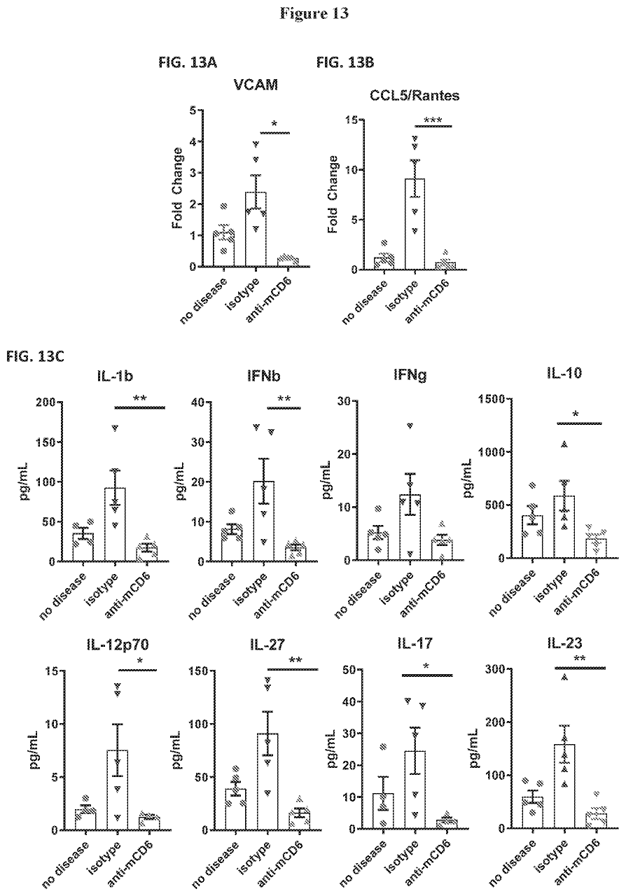

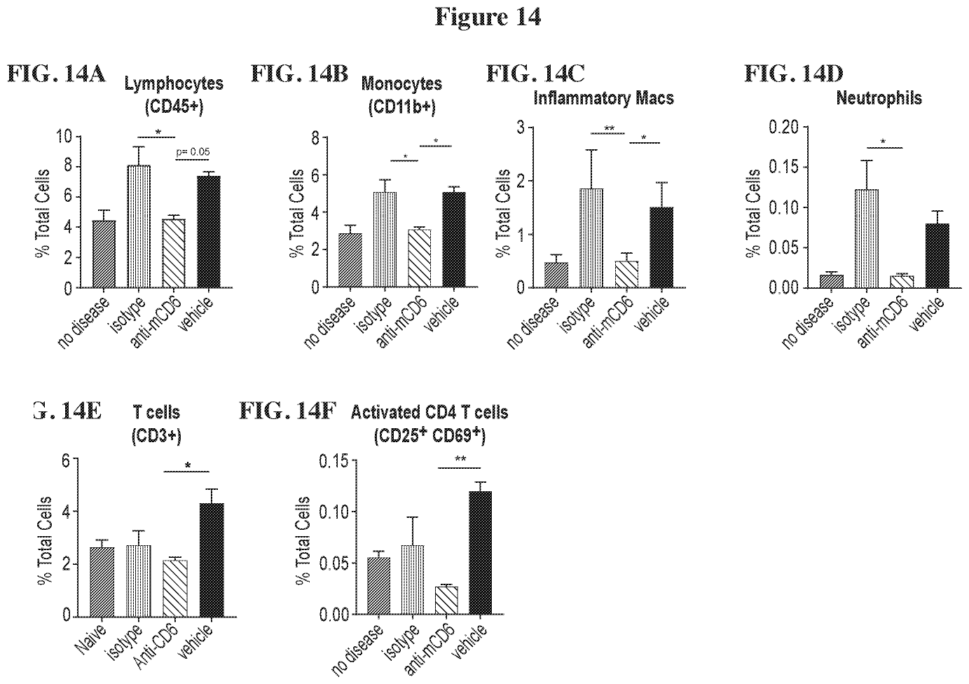

RT-PCR for renal cytokine levels was performed to determine the effect of CD6 blockade on renal inflammation, and these experiments revealed a less inflammatory milieu of cytokines in the kidneys of treated mice as compared to control sick mice, with significantly decreased expression levels of inflammatory markers VCAM (FIG. 13A) and RANTES (FIG. 13B) and increased levels of anti-inflammatory IL-10 (FIG. 13C). Flow cytometry was performed on kidneys to assess the effect of anti-mCD6 treatment on immune cell infiltration. We noted an overall decrease in immune cell accumulation (FIG. 14A, CD45+) in anti-CD6 treated mice vs both isotype and vehicle control mice. Further analysis showed decreases in inflammatory myeloid cells (FIG. 14B to FIG. 14D) and in T cell populations (FIG. 14E to FIG. 14F). T cells were significantly decreased (FIG. 14E), with a significant difference noticed in activated CD4 (CD25+CD69+) cells (FIG. 14F).

CD6 blockade improves kidney function in this mouse model via reductions in renal inflammatory cytokine expression and immune infiltration of myeloid and T cells in the kidney.

These results highlight the CD6-ALCAM pathway as a promising therapeutic option which is more selective than the immunosuppressive therapies currently offered.

Thus, the forgoing experiments demonstrate that inhibition of the CD6-ALCAM pathway with an anti-CD6 treatment (e.g., after diagnosis according to the methods disclosed herein) ameliorates the nephritis associated with nephrotoxic antibody administration, an inducible model of lupus nephritis.

Taken together, the studies reported herein provide strong support that LN may be diagnosed and/or that the LN activity state and disease progression may be monitored by analyzing the presence and abundance of ALCAM or CD6 protein in urine and/or mRNA expression in urinary lymphocytes, and these studies further suggest that a targeted CD6-ALCAM therapy, such as itolizumab, may be a promising candidate for the treatment of LN.

### Example 6

### CD6-ALCAM Pathway is Active in the NZB/W F1 and B6.Sle1yaa Models of SLE

Background/Purpose: To facilitate further study into the effect of CD6 blockade on serum levels of ALCAM we first sought to identify murine models of SLE that recapitulate the increases in urine ALCAM levels that we observed in human patients.

To that end, we first examined serum ALCAM levels in the NZB/W F1 and B6.Sle1yaa models of SLE.

NZB/W F1 Model. NZB/W F1 mice are the F1 hybrid between the New Zealand Black (NZB) and New Zealand White (NZW) strains. NZBWF1/J mice develop an autoimmune disease resembling human systemic lupus erythematosus. Similar to human disease, autoimmunity develops primarily in female animals and is characterized by high levels of antinuclear antibodies, hemolytic anemia, proteinuria, and progressive immune complex glomerulonephritis. The major cause of death in the NZB/W F1 female is chronic glomerulonephritis with heavy mesangial deposits before 5 months of age, tubular cast formation, proliferation of glomerular cells, prominent crescent formation, and a significant periglomerular and interstitial monocytic infiltrate. Extraglomerular renal deposits of IgG2a and C3 are present in the peritubular tissue and arterioles, and increase in frequency with age.

B6.Sle1yaa model. B6.Sle1yaa males are C57BL/6J-congenic animals carrying the systemic lupus erythematosus susceptibility 1 quantitative trait locus from NZM2410/Aeg inbred mice and the mutant Yaa-containing Y chromosome from BXSB/MpJ inbred mice. B6.Sle1yaa males develop spontaneous lupus-like autoimmune syndrome with numerous immunological aberrations. Specifically, mortality in B6.Sle1yaa males starts at ˜12-15 weeks of age with 50% lethality by ˜30-38 weeks of age. In addition, B6.Sle1yaa males exhibit severe kidney pathology characterized by hyalanized end-stage disease in most kidney glomeruli. Significant levels of auto-antibodies are detectable by 6-8 weeks, and IgG auto-antibodies against dsDNA and kidney glomerular antigens increase dramatically with onset of severe glomerulonephritis around 4-6 months. The CD4+ T cell lineage is dysregulated in B6.Sle1yaa males: early and progressive CD4+ T cell activation leads to increased IFN γ-secreting cells and, eventually, to a chronic-activation induced replicative senescence.

Based upon the association between urine ALCAM and LN in human disease, we assessed the level of urine ALCAM within the context of these murine model of SLE, and then subsequently targeted this signaling axis to determine its role in disease.

We first analyzed urine ALCAM expression in the NZB/W F1 female mice to confirm the overexpression observed in human samples is recapitulated in this model. In an initial experiment, urine was collected from 6 month old and 12 month old mice and ALCAM levels assessed by ELISA.

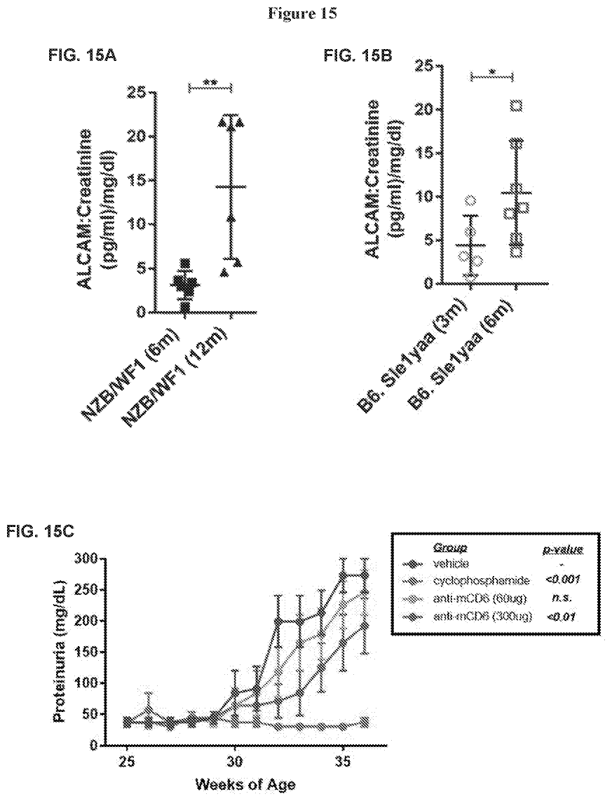

The data in the NZB/W F1 model are shown in FIG. 15A, which demonstrates significant increases in serum ALCAM levels (normalized to urine Creatinine level) post-disease onset (12 months) as compared with serum ALCAM levels (normalized to urine Creatinine level) pre-disease onset (≤6 months).

We next analyzed urine ALCAM expression in the B6.Sle1yaa model. Urine was collected from 3 month old and 6 month old mice and ALCAM levels assessed by ELISA. The data in the B6.Sle1yaa model are shown in FIG. 15B, which demonstrates significant increases in serum ALCAM levels (normalized to urine Creatinine level) post-disease onset (6 months) as compared with serum ALCAM levels (normalized to urine Creatinine level) pre-disease onset (3 months).

The data in the NZB/W F1 and B6.Sle1yaa models both mimic the increase in urine ALCAM levels associated with renal disease that was observed in human patients. These results indicate that these murine models are appropriate for further mechanistic exploration of urine ALCAM levels and the effect of CD6 blockade on disease progression and ALCAM/CD6 levels in urine.

To that end, in a follow-up experiment, we tested whether CD6 blockade in the NZB/W F1 female mouse model was able to affect disease progression. Table 3 shows the study design of the experiment using the NZB/W F1 model.

TABLE 3
Study design of CD6 blockade experiment using the NZB/W F1 murine model

| | | # | | Dosing | Dosing | Dosing | Dosing |
|---|---|---|---|---|---|---|---|
| Group | Strain | Mice | Treatment | Concentration | Route | Frequency | Duration |
| 1 | NZBWF 1 | 10 | Vehicle | N/A | IP | Twice Weekly | 10 Weeks |
| 2 | NZBWF 1 | 10 | *Cyclophos | 25 mg/kg | IP | Twice Weekly | 10 Weeks |
| 3 | NZBWF 1 | 10 | 10D12 | 60 μg | IP | Twice Weekly | 10 Weeks |
| 4 | NZBWF 1 | 10 | 10D12 | 300 μg | IP | Twice Weekly | 10 Weeks |

*Cyclophos = Cyclophosphamide

Briefly, female NZB/W F1 mice (age: 26 weeks) were treated intraperitoneally twice weekly with either anti-CD6 antibody (10D12, 60 or 300 ug/dose), cyclophosphamide (25 mg/kg), or vehicle. Proteinuria and weight was assessed weekly.

The results of this experiment are shown in FIG. 15C. Treatment of these NZB/W F1 female mice with anti-mCD6 antibody resulted in significant decreases in proteinuria, an important measure of renal function. Thus, changes in urine ALCAM in the NZB/W F1 and B6.Sle1yaa models are indicative of SLE disease progression and may be used as a biomarker useful for determining when treatment with an anti-mCD6 antibody will be effective.

### REFERENCES

The contents of all references cited herein are hereby incorporated by reference herein for all purposes.
1. Gangemi et al., Anti-T12, A n Anti-CD6 Monoclonal-Anti body, Can Activate Human Lymphocytes-t J. Immunol. 1989, 143:2439-2447.
2. Bott et al., Activation of Human T-cells Through CD6—Functional—Effects of A Novel Anti-CD6 Monoclonal—Anti body and Definition of 4 Epitopes of The CD6 Glycoprotein, Int Immunol. 1993, 7:783-792.
3. Morimoto et al., 2h1—A Novel Antigen Involved in Lymphocyte-T Triggering, J. Immunol. 1988, 140:2165-2170.
4. Osorio et al. The Anti-CD6 mAb, iOR-tl, Defined a New Epitope on The Human CDS Molecule That Induces Greater Responsiveness in T-cell Receptor/CD 3-Mediated T-cell Proliferation, Cell. Immunol. 1994, 154: 123-133.
5. Swack et al., Structural Characterization of CD6—Properties of 2 Distinct Epitopes Involved In T-cell Activation Structural Characterization of CD6—Properties of 2 Distinct Epitopes Involved In T-cell Activation, Mol. Immunol. 1989 26:1037-1049.
6. Swack et al., Biosynthesis And Posttranslational Modification of CD6, a T-cell Signal-Transducing Molecule, J. Biol. Chem 1991, 266:7137-7143.
7. Cardenas et al., Phosphoryiation-Dephosphorylation of The CD6 Glycoprotein Renders 2 Isoforms of 130 and 105 Kilodaltons—Effect of Serum and Protein-Kinase-C Activators, J. Immunol. 1990, 145:1450-1455.
8. Wee et al., Tyrosine Phosphorylation of CD6 By Stimulation of CDS—Augmentation By The CD4 and CD2 Coreceptors, J. Exp. Med. 1993, 177:219-223.
9. Patel, et al., Identification And Characterization of A 100-Kd Ligand For CD6 On Human Thymic Epithelial-Cells, J. Exp. Med. 1995. 181:1563-1568.
10. Bowen et al., Cloning, Mapping, And Characterization of Activated Leukocyte—Cell Adhesion molecule (ALCAM), a CD6 Ligand, J. Exp. Med 1995, 181:2213-2220.
11. Whitney, et. al., The Membrane-Proximal Scavenger Receptor Cysteine-Rich Domain of CD6 Contains The Activated Leukocyte Cell—Adhesion Molecule-Binding Site, J Biol. Chem 1995, 270: 18187-18190.
12. Gimferrer I, Relevance of CD6-mediated interactions in T-cell activation and proliferation, J Immunol 2004. 173: 2262-2270.
13. Liu, K. and E. K. Wakeland, Delineation of the pathogenesis of systemic lupus erythematosus by using murine models. Adv Exp Med Biol, 2001. 490: p. 1-6.
14. Arrias M R, et al., CD6 binds to pathogen-associated molecular patterns and protects from LPS-induced septic shock. Proc Natl Acad Sci USA (2007) 104:11724-9.
15. Aibar J, et al. Pattern of soluble CD5 and CD6 lymphocyte receptors in critically ill patients with septic syndromes. J Crit Care (2015) 30:914-9.
16. Ramos-Casals M, et al. High circulating levels of soluble scavenger receptors (sCD5 and sCD6) in patients with primary Sjogren's syndrome. Rheumatology (2001) 40:1056-9.
17. Kim, et al., Activated Leukocyte Cell Adhesion Molecule Stimulates the T-Cell Response in Allergic Asthma, Am J Respir Crit Care Med. 2018 Apr. 15; 197(8):994-1008.
18. Egloff, et al., Shed urinary ALCAM is an independent prognostic biomarker of three-year overall survival after cystectomy in patients with bladder cancer, Oncotarget. 2017 Jan. 3; 8(1):722-741.
19. Habazi, D. et al., ALCAM and VCAM-1 as potential urinary biomarkers in Caucasian lupus patients, J Immunol May 1, 2018, 200 (1 Supplement) 175.17.

SEQUENCES:
EQ001 VH amino acid sequence:
(SEQ ID NO: 1)
EVQLVESGGGLVKPGGSLKLSCAASGFKFSRYAMSWVRQAPGKRLEW
VATISSGGSYIYYPDSVKGRFTISRDNVKNTLYLQMSSLRSEDTAMY
YCARRDYDLDYFDSWGQGTLVTVSS
EQ001 VK amino acid sequence:
(SEQ ID NO: 2)
DIQMTQSPSSLSASVGDRVTITCKASRDIRSYLTWYQQKPGKAPKTL
IYYATSLADGVPSRFSGSGSGQDYSLTISSLESDDTATYYCLQHGES
PFTLGSGTKLEIK
EQ001 VH nucleotide (DNA) sequence:
(SEQ ID NO: 3)
GAAGTGCAGCTGGTGGAGTCTGGGGGAGGCTTAGTGAAGCCTGGAGG
GTCCCTGAAACTCTCCTGTGCAGCCTCTGGATTCAAGTTTAGTAGAT
ATGCCATGTCTTGGGTTCGCCAGGCTCCGGGGAAGAGGCTGGAGTGG
GTCGCAACCATTAGTAGTGGTGGTAGTTACATCTACTATCCAGACAG
TGTGAAGGGTCGATTCACCATCTCCAGAGACAATGTCAAGAACACCC
TGTATCTGCAAATGAGCAGTCTGAGGTCTGAGGACACGGCCATGTAT
TACTGTGCAAGACGAGATTACGACCTGGACTACTTTGACTCCTGGGG
CCAAGGCACCCTTGTCACCGTCTCCTCA
EQ001 VK nucleotide (DNA) sequence:
(SEQ ID NO: 4)
GACATCCAGATGACCCAGTCTCCATCCTCCCTGTCTGCATCGGTGGG
AGACAGAGTCACTATCACTTGCAAGGCGAGTCGGGACATTAGAAGCT
ATTTAACCTGGTACCAGCAGAAACCAGGGAAAGCTCCTAAGACCCTG
ATCTATTATGCAACAAGCTTGGCAGATGGGGTCCCGTCGAGATTCAG
TGGCAGTGGATCTGGGCAAGATTATTCTCTCACCATCAGCAGCCTGG
AGTCTGACGATACAGCAACTTACTACTGTCTACAACATGGTGAGAGT
CCATTCACGCTCGGCTCGGGGACCAAGCTGGAAATCAAA
EQ001 heavy chain CDR1 amino acid sequence:
(SEQ ID NO: 5)
GFKFSRYAMS;
EQ001 heavy chain CDR2 amino acid sequence:
(SEQ ID NO: 6)
TISSGGSYIYYPDSVKG;
EQ001 heavy chain CDR3 amino acid sequence:
(SEQ ID NO: 7)
RDYDLDYFDS
EQ001 light chain CDR1 amino acid sequence:
(SEQ ID NO: 8)
KASRDIRSYLT
EQ001 light chain CDR2 amino acid sequence:
(SEQ ID NO: 9)
YATSLAD
EQ001 light chain CDR3 amino acid sequence:
(SEQ ID NO: 10)
LQHGESP

### EQUIVALENTS

While the present invention has been described in conjunction with the specific embodiments set forth above, many alternatives, modifications and other variations thereof will be apparent to those of ordinary skill in the art. All such alternatives, modifications and variations are intended to fall within the spirit and scope of the present invention. All of the U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified, if necessary to employ concepts of the various patents, application and publications to provide yet further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method for identifying whether a subject has a form of lupus nephritis that is sensitive to CD6-ALCAM pathway inhibition, the method comprising determining whether the subject exhibits an elevated level of soluble CD6 and/or ALCAM protein.

2. A method for treating lupus nephritis with a CD6-ALCAM pathway inhibitor, the method comprising:
a. determining whether a biological sample obtained from a subject having or suspected of having lupus nephritis contains an elevated level of soluble CD6 and/or ALCAM protein; and
b. administering to the subject a CD6-ALCAM pathway inhibitor if the biological sample contains an elevated level of soluble CD6 and/or ALCAM protein.

3. A method for using a CD6-ALCAM pathway inhibitor to treat a subject with lupus nephritis, the method comprising the steps of:
a. determining whether the subject exhibits elevated levels of soluble CD6 and/or ALCAM protein; and
b. administering to the subject the CD6-ALCAM pathway inhibitor if the subject exhibits elevated levels of soluble CD6 and/or ALCAM protein.

4. A method for treating a subject with a CD6-ALCAM pathway inhibitor, wherein the subject has lupus nephritis, the method comprising the steps of:
a. determining whether the subject has a CD6-ALCAM pathway inhibitor-sensitive disease by:
i. obtaining or having obtained a biological sample from the subject; and
ii. performing or having performed an assay on the biological sample to determine if the sample exhibits an elevated level of soluble CD6 and/or ALCAM protein; and
b. administering the CD6-ALCAM pathway inhibitor to the subject if the subject has elevated soluble CD6 and/or ALCAM protein.

5. A method for identifying whether a subject has an inflammatory or autoimmune disease that is sensitive to CD6-ALCAM pathway inhibition, the method comprising determining whether the subject exhibits an elevated level of soluble CD6 and/or ALCAM protein.

6. A method for treating an inflammatory or autoimmune disease with a CD6-ALCAM pathway inhibitor, the method comprising:
a. determining whether a biological sample obtained from a subject having or suspected of having inflammatory or autoimmune disease contains an elevated level of soluble CD6 and/or ALCAM protein; and
b. administering to the subject a CD6-ALCAM pathway inhibitor if the biological sample contains an elevated level of soluble CD6 and/or ALCAM protein.

7. A method for using a CD6-ALCAM pathway inhibitor to treat a subject with an inflammatory or autoimmune disease, the method comprising the steps of:
a. determining whether the subject exhibits elevated soluble CD6 and/or ALCAM protein; and
b. administering to the subject the CD6-ALCAM pathway inhibitor if the subject exhibits elevated soluble CD6 and/or ALCAM protein.

8. A method for treating a subject with a CD6-ALCAM pathway inhibitor, wherein the subject has inflammatory or autoimmune disease, the method comprising the steps of:
a. determining whether the subject has a CD6-ALCAM pathway inhibitor-sensitive disease by:
i. obtaining or having obtained a biological sample from the subject; and
ii. performing or having performed an assay on the biological sample to determine if the sample exhibits an elevated level of soluble CD6 and/or ALCAM protein; and
b. administering the CD6-ALCAM pathway inhibitor to the subject if the subject has elevated soluble CD6 and/or ALCAM protein.

9. The method of any one of claims 1-8, wherein the CD6-ALCAM pathway inhibitor is EQ001.

10. The method of any one of claims 1-8, wherein the CD6-ALCAM pathway inhibitor is an anti-CD6 antibody, or the antigen binding fragment thereof.

11. The method of claim 10, wherein the anti-CD6 antibody, or the antigen binding fragment thereof, is a humanized antibody.

12. The method of claim 10, wherein the anti-CD6 antibody, or the antigen binding fragment thereof, binds to domain 1 or 3 on CD6.

13. The method of claim 10, wherein the anti-CD6 antibody, or the antigen binding fragment thereof, binds to domain 3 on CD6.

14. The method of claim 10, wherein the anti-CD6 antibody, or the antigen binding fragment thereof, is selected from the group consisting of: EQ001, ALZUMAb, UMCD6 mAb, Itolizumab, T1h, an anti-CD6 antibody described on Table 1, and an anti-CD6 antibody disclosed herein.

15. The method of claim 10, wherein the anti-CD6 monoclonal antibody is an antibody produced by secreting hybridoma IOR-T1A deposited with the ECACC as deposit No. ECACC 96112640; an antibody having the same sequence as said antibody produced by said secreting hybridoma; or an antibody having the same CDR sequences of said antibody produced by said secreting hybridoma.

16. The method of claim 10, wherein the antigen binding fragment is selected from an Fv, Fab, CDR1, CDR2, CDR3, combination of CDRs, variable region, heavy chain(s), and light chain(s).

17. The method of any one of claims 10-16, wherein the anti-CD6 antibody, or the antigen binding fragment thereof, comprises one or more CDR sequence selected from SEQ ID NOS: 5-10.

18. The method of any one of claims 10-17, wherein the anti-CD6 antibody, or the antigen binding fragment thereof, comprises heavy and light chain variable regions comprising amino acid sequences as set forth in SEQ ID NOs: 1 and 2.

19. The method of claim 18, wherein SEQ ID NOs: 1 and 2 are encoded by SEQ ID NOs: 3 and 4 respectively.

20. The method of any one of claims 10-17, wherein the anti-CD6 antibody, or the antigen binding fragment thereof, comprises a VH sequence that is at least 80%, 85%, 90%, or 95% identical to the amino acid sequence as set forth in SEQ ID NO: 1.

21. The method of any one of claims 10-17, wherein the anti-CD6 antibody, or the antigen binding fragment thereof, comprises a VK sequence that is at least 80%, 85%, 90%, or 95% identical to the amino acid sequence as set forth in SEQ ID NO: 2.

22. The method of any one of claims 10-17, wherein the anti-CD6 antibody, or the antigen binding fragment thereof, comprises a VH sequence that is at least 80% identical to the amino acid sequence as set forth in SEQ ID NO: 1 and a VK sequence that is at least 80% identical to the amino acid sequence as set forth in SEQ ID NO: 2.

23. The method of any one of claims 1-22, wherein the subject exhibits the elevated level of soluble CD6 and/or ALCAM protein in a sample selected from blood, serum, urine, sputum, CSF, BALF, and stool.

24. The method of any one of claims 1-22, wherein the subject exhibits the elevated level of soluble CD6 and/or ALCAM protein in urine.

25. The method of any one of claims 5-24, wherein the subject has lupus nephritis.

26. The method of any one of claims 1-4 and 25, wherein the soluble CD6 and/or ALCAM protein is elevated in the subject as compared to an individual that does not have lupus nephritis.

27. The method of any one of claims 5-24, wherein the soluble CD6 and/or ALCAM protein is elevated in the subject as compared to an individual that does not have the inflammatory or autoimmune disease.

28. The method of any one of the preceding claims, wherein the level of soluble CD6 and/or ALCAM protein is determined in a first and one or more second sample from the subject.

29. The method of claim 28, wherein the level of soluble CD6 and/or ALCAM protein is elevated in a second sample as compared to the level of soluble CD6 and/or ALCAM protein that was present in the first sample.

30. The method of claim 29, wherein the elevated level of soluble CD6 and/or ALCAM protein in the second sample indicates active disease in the subject.

31. The method of claim 28, wherein a decrease in the level of soluble CD6 and/or ALCAM protein in the second sample indicates transition from an active disease to a passive disease in the subject.

32. The method of claim 29 or 30, wherein a threshold increase in the level of soluble CD6 and/or ALCAM protein in the second sample as compared to the first sample indicates transition from a passive disease to an active disease in the subject.

33. The method of claim 28, wherein the level of soluble CD6 and/or ALCAM protein is not elevated in a second sample as compared to the level of soluble CD6 and/or ALCAM protein that was present in the first sample.

34. The method of claim 33, wherein the level of soluble CD6 and/or ALCAM protein in the second sample indicates that the subject does not have lupus nephritis or any inflammatory or autoimmune disease.

35. The method of claim 28, wherein the level of CD6 and/or ALCAM is measured in a plurality of second samples obtained from the subject over a time course of days, weeks, months, or years.

36. The method of any of the preceding claims, wherein the level of CD6 and/or ALCAM protein is detected using a method selected from single-plex ELISA;
multiplex ELISA, bead-based immunocapture with FACs-based detection; bead-based immunocapture with ELISA-based detection; bead-based immunocapture with chemiluminescent-based detection; meso-scale diagnostic (MSD);
quantitative western blot; high performance liquid chromatography (HPLC); and a combination thereof.

37. The method of any one of the preceding claims, wherein the CD6 and/or ALCAM protein that is detected is a full length protein.

38. The method of any one of claims 1-37, wherein the CD6 and/or ALCAM protein that is detected is a fragment of the full length protein.

39. The method of claim 38, wherein the fragment of the full length CD6 protein that is detected comprises the entire extracellular domain of CD6, or a portion of the extracellular domain of CD6.

40. The method of any one of the preceding claims, comprising administering to the subject EQ001.

41. The method of claim 40, wherein the method further comprises administering an additional therapeutic agent.

42. The method of claim 41, wherein the additional therapeutic agent is a steroid or an immunosuppressant.

43. The method of claim 42, wherein the steroid is a corticosteroid.

44. The method of claim 43, wherein the corticosteroid is prednisone.

45. The method of claim 41, wherein the agent is selected from mycophenolate and cyclophosphamide.

46. A method of predicting the prognosis of a subject with lupus nephritis, the method comprising the steps of:
i. obtaining or having obtained a plurality of biological samples from the subject over a time course of days, weeks, months or years; and
ii. performing or having performed an assay on each of the biological samples to determine if there is a change over time in the level of soluble CD6 and/or ALCAM proteins that are present in the sample;
wherein
b. if the sample exhibits an increase in the level of soluble CD6 and/or ALCAM protein over time, then the prognosis is determined to be poor;
c. if the sample exhibits no change in the level of soluble CD6 and/or ALCAM protein over time, then the prognosis is determined to be neutral; and
d. if the sample exhibits a decrease in the level of soluble CD6 and/or ALCAM protein over time, then the prognosis is determined to be good.

47. A method of predicting the prognosis of a subject with an inflammatory or autoimmune disease, the method comprising the steps of:
i. obtaining or having obtained a plurality of biological samples from the subject over a time course of days, weeks, months or years; and
ii. performing or having performed an assay on each of the biological samples to determine if there is a change over time in the level of soluble CD6 and/or ALCAM proteins that are present in the sample; wherein
b. if the sample exhibits an increase in the level of soluble CD6 and/or ALCAM protein over time, then the prognosis is determined to be poor;
c. if the sample exhibits no change in the level of soluble CD6 and/or ALCAM protein over time, then the prognosis is determined to be neutral; and
d. if the sample exhibits a decrease in the level of soluble CD6 and/or ALCAM protein over time, then the prognosis is determined to be good.

48. A method of determining whether a subject has active lupus nephritis comprising
a. determining a first concentration of soluble CD6 and/or ALCAM protein present in a sample from the subject;
b. determining a second concentration, or average concentration, of soluble CD6 and/or ALCAM protein present in a similar sample from a control person, or a population of control persons, respectively, that do not have active lupus nephritis; and
c. determining that the subject has active nephritis if the first concentration is greater than the second concentration.

49. A method of determining whether a subject has active inflammatory or autoimmune disease comprising
a. determining a first concentration of soluble CD6 and/or ALCAM protein present in a sample from the subject;
b. determining a second concentration, or average concentration, of soluble CD6 and/or ALCAM protein present in a similar sample from a control person, or a population of control persons, respectively, that do not have active lupus nephritis; and
c. determining that the subject has active nephritis if the first concentration is greater than the second concentration.

50. A method of determining whether a subject has transitioned from inactive lupus nephritis to active lupus nephritis comprising
a. determining a first concentration of soluble CD6 and/or ALCAM protein present in a first sample from the subject; wherein the first sample is obtained from the subject when the subject has inactive lupus nephritis;
b. determining a second concentration of soluble CD6 and/or ALCAM protein present in one or more second samples from the subject; wherein each second sample is obtained from the subject after the first sample was obtained; and
c. determining that the subject has active lupus nephritis or is transitioning into active nephritis if the second concentration of soluble CD6 and/or ALCAM protein is greater than the first concentration.

51. A method of determining whether a subject has transitioned from inactive inflammatory or autoimmune disease to active inflammatory or autoimmune disease comprising
a. determining a first concentration of soluble CD6 and/or ALCAM protein present in a first sample from the subject; wherein the first sample is obtained from the subject when the subject has inactive lupus nephritis;
b. determining a second concentration of soluble CD6 and/or ALCAM protein present in one or more second samples from the subject; wherein each second sample is obtained from the subject after the first sample was obtained; and
c. determining that the subject has active lupus nephritis or is transitioning into active nephritis if the second concentration of soluble CD6 and/or ALCAM protein is greater than the first concentration.

52. The method of any one of claims 48-51, further comprising administering to the subject EQ001 if the subject has active LN or is transitioning into active LN.

53. The method of claim 52, wherein the method further comprises administering an additional therapeutic agent.

54. The method of claim 53, wherein the additional therapeutic agent is a steroid or an immunosuppressant.

55. The method of claim 54, wherein the steroid is a corticosteroid.

56. The method of claim 55, wherein the corticosteroid is prednisone.

57. The method of claim 53, wherein the agent is selected from mycophenolate and cyclophosphamide.

58. The method of any one of the preceding claims, wherein the CD6-ALCAM pathway inhibitor is an anti-CD6 monoclonal antibody that is administered by parenteral delivery.

59. The method of any one of the preceding claims, wherein the CD6-ALCAM pathway inhibitor is an anti-CD6 monoclonal antibody that is administered with a pharmaceutically acceptable carrier.

60. The method of any one of the preceding claims, wherein the anti-CD6 antibody is a humanized antibody.
